(19) United States
(12) Patent Application Publication
(10) Pub. No.: US 2005/0112108 A1
(43) Pub. Date: May 26, 2005

(54) Transcriptional regulator of genes involved in the control of cell growth or cell proliferation. Use of said regulator as a therapeutic or diagnostic agent

(76) Inventor: Michael Tovey, Paris (FR)

Correspondence Address: BIRCH STEWART KOLASCH & BIRCH, PO BOX 747, FALLS CHURCH, VA, 22040-0747, US

(21) Appl. No.: 10/727,569

(22) Filed: Dec. 5, 2003

Related U.S. Application Data
(63) Continuation of application No. PCT/EP02/07064, filed on Jun. 6, 2002.

(30) Foreign Application Priority Data
Jun. 7, 2001 (EP) 01401476.5

(51) Int. Cl. A61K 48/00; C12N 15/85

(52) U.S. Cl. 424/93.21; 435/455

(57) ABSTRACT

Constitutive expression of the tumor suppressor genes p53 and IRF1 (interferon regulatory factor 1) is required for the maintenance of the cellular growth control during genotoxic damage or abberant proliferation. A transcriptional regulatory element within the promoter region of both the p53 and IRF-1 genes which binds a positive transcription factor, denoted GAAP-1 or GAAP-2 (IRF1 p53 common sequence binding factor). The invention relates to the identification and cloning of the GAAP-1 (or GAAP-2 cDNA and to the identification of the encoded protein corresponding to the 75 kDa product of the alternatively sliced PRDII-BF1 messenger RNA precursor.

The present invention relates to a transcriptional regulator of genes involved in control of cell growth and cell proliferation and relates to the use of the gene for the preparation of therapeutic or diagnostic means.

The development of cells toward a malignant state is controlled in the human body by numerous parameters including the expression of specific genes, some of which are constitutively expressed in cells. Considerable effort has been directed in the past years towards the identification of the mechanisms of malignant transformation of cells particularly with the aim of identifying new therapeutic approaches and tools including products, in order to remedy to said transformation toward a malignant cell or in order to stop progression of such a state toward the formation of tumors and in some instances, in order to reverse the malignant state towards a normal state.

In the field of cancer research, some genes have been identified in cells, the regulation of which is affected during the transformation of cells toward a malignant state. Such genes include “anti-oncogenes”, some of which have been identified, cloned and sequenced. Among these genes, p53 and IRF1 (Interferon Regulatory Factor 1) have been well characterised and it has been shown that their maintenance in cellular growth control during genotoxic damage or aberrant proliferation is required in order to prevent the development of a malignant cell.

Starting from the observation that said p53 and IRF1 genes are involved in preventing the transformation of cells toward a malignant state, the inventors have studied their regulation and have found ways to interact with the control functions controlled by these genes.

The present invention offers means which can interfere with the transformation of cells toward a malignant state, or which can be used to control the spreading of such cells including by enabling their apoptosis, said means being at least to some extent capable of overcoming some drawbacks and especially the insufficient level of activity of the p53 or IRF1 genes observed when these genes are used to design therapeutic protocols.

The invention indeed offers the possibility to control the transcription of a broad spectrum of genes having functions in the maintenance of normal growth or differenciation of cells.

A publication (Lallemand C. et al, 1997) has disclosed the presence in the promoter regions of both the IRF1 and p53 anti-oncogenes of a sequence denoted “IRF1 p53 common sequence” (IPCS) which markedly increases the transcriptional activity of a reporter gene placed under the control of a heterologous promoter in transfected U937 cells,

According to this publication, the IPCS sequence is defined as a degenerated motif replying to the sequence AAATGRYKKCMMS (IUAP code) which sequence has been shown according to said publication to bind a nuclear factor denoted IPCS-binding factor (IPCS-BF). Said IPCS-BF factor is composed of a single polypeptide of 26 kDa which is present constitutively in nuclear extracts of U937 cells and Peripheral Blood Mononuclear cells from healthy donors. According to the publication of Lallemand C. et al, it is believed that the transcriptional regulation of the p53 and IRF1 genes is influenced by the binding of said IPCS-BF factor, to the IPCS sequence.

The inventors have now identified novel related transcription factors including two members originating from the expression of the same PRDII-BF2 gene, denoted GAAP-1 and GAAP-2 (Gatekeeper of Apoptosis Activating Proteins) which influence the transcription regulation of at least the p53 and IRF1 genes. According to the present invention, these GAAP-1 and GAAP-2 factors bind specifically to the IPCS sequence found in the promoter regions of both p53 and IRF1 genes. GAAP-1 corresponds to a protein of around 75 kDa which is an alternative splice product of a known gene, PRDII-BF1 (Fan C. M. and Maniatis, T., 1990; Muchardt C. et al, 1992).

GAAP-2 is another alternative splice product of the PRDII-BF1 gene. It contains 23 additional amino acid residues with respect to the amino acid sequence of GAAP-1.

The PRDII-BF1 gene has been disclosed as a gene encoding a 300 kDa cellular protein with two widely spaced zinc fingers. It has also been presented as enabling the alternative production of spliced gene products encoding respectively proteins of 200 and 70 kDa. The spliced forms of the PRDII-BFI cDNA have been disclosed in the above-cited publication of Muchardt C. et al, showing that the short transcript is devoid of the nucleotide sequence of the PRDII-BF1 gene corresponding to exon IV.

Accordingly, this short transcript gives rise to a cDNA sequence comprising as a coding sequence, the exon I, II, III, V, VI, VII, VIII and IX of the PRDII-BF1 gene and thus, lacks the sequence comprised between positions 416 and 6394 of the gene as disclosed in Muchardt et al 1992 with reference to Fan et al 1990.

The invention identifies novel transcription factors including GAAP-1 and GAAP-2 interacting with a specific sequence present in the regulation region of several genes and therefore opens a new way for the definition of a means useful in a therapeutic, diagnostic or prognostic approach of various pathologies including those related to malignant cells. The above-mentioned factors GAAP-1 and GAAP-2 both represent alternative splice products of the same gene, i.e., PRDII-BF1, and can be regarded as variants of the same gene.

The invention thus relates to the use of a nucleotide sequence selected among:
(i) a nucleotide sequence comprising the DNA sequence identified under No. 1, and represented on FIG. 14A, or under No. 3 and represented on FIG. 14B;
(ii) a nucleotide sequence encoding a polypeptide having the amino-acid sequence identified under No. 2, and represented on FIG. 15A or encoding a polypeptide having the amino-acid sequence identified under No. 4 and represented on FIG. 15B;
(iii) a nucleotide sequence comprising the DNA sequence identified under No. 5, and represented on FIG. 14C;
(iv) a nucleotide sequence encoding a polypeptide having the amino-acid sequence identified under No. 6, and represented on FIG. 15C;
(v) a nucleotide sequence derived from sequence defined under (i), (ii), (iii) or (iv) wherein said sequence is modified particularly by deletion, addition or substitution of one or more nucleotides providing that the resulting nucleotide sequence encodes a polypeptide capable of binding a nucleotide sequence designated IPCS which comprises the DNA sequence AAATGNNNNC, wherein N means any nucleotide (G, A, C or T(U))
   for the expression in a determined eucaryotic cell, of a polypeptide capable of interacting with said nucleotide sequence designated IPCS and capable of acting as a positive transcriptional factor for the transcription of a nucleotide sequence placed under the control of said IPCS sequence and present in said eucaryotic cell.

The above nucleotide sequence encodes the transcription factor of the invention, in conditions enabling its interaction and especially its binding with the identified IPCS sequence.

The IPCS sequence has been defined in the prior publication of Lallemand C et al 1997, and appears as a consensus sequence of 6 nucleotide residues in a sequence of generally 10 nucleotides. The sequence is disclosed by reference to the IUPAC code and therefore the positions GNNNN can respectively correspond to nucleotide guanine and to any nucleotides (G, A, C or T(U)).

Particular IPCS sequences have been found in the p53 and IRF1 genes, which have the following DNA sequence: AAAATGATTTCCAC (for the p53 gene) and GAAATGACGGCACG (for the IRF1 gene). This sequence has also been identified in other anti-oncogenes such as Rb, p21 (WAF1), p27. wt1, bax, TNF receptor and FAS genes.

These IPCS sequences are described hereafter by reference to the gene containing the same.

| | | | |
|---|---|---|---|
| Gene | Pos. | Sequence IPCS | Ref. genebank |

| | | | |
|---|---|---|---|
| Rb | 680 | aaatgtattc | L11910 |
| P21 | 1138 | aaatgtattc | U24170 |
| P21 | 1876 | aaatgaaaac | U24170 |
| P21 | 2031 | aaatggtgac | U24170 |
| P21 | 4314 | aaatgtgtcc | U24170 |
| P27 | 292 | aaatggcaac | AB003688 |
| Wt1 | 1828 | aaatgggctc | X74840 |
| Bax | 679 | aaatggtgcc | U17193 |
| Bax | 694 | aaatgaaggc | U17193 |
| TNFr | 946 | aaatgaacac | U53483 |
| FAS | 580 | Aaatgtcaac | U31968 |

The DNA sequences identified under No.1, No. 3 and No. 5 are the sequences presented respectively in FIGS. 14A, 14B and 14C and designated respectively GAAP-1 and GAAP-2.

The aminoacid sequences identified under No 2., No. 4 and No. 6 are presented respectively in FIGS. 15A, 15B and 15C and designated respectively GAAP-1 and GAAP-2.

The theoretical molecular weight for GAAP-1 as disclosed on FIG. 15B and GAAP-2, as disclosed on FIG. 15C are respectively 75.7 and 77.5 kDa.

The deletion, insertion or substitution in the above defined nucleotide sequences of the invention, starting from the DNA sequence encoding GAAP-1, include point mutation or mutation of several nucleotides provided the properties of the encoded polypeptide in accordance with the present invention, are retained in the mutated sequence. According to a preferred embodiment such a mutated sequence retains the zinc finger domains of the native sequence.

A particular mutated sequence encompasses the DNA sequence encoding GAAP-2 represented on FIG. 15C. The GAAP-1 amino-acid sequence represented on FIG. 15B is a variant of GAAP-1 as shown on FIG. 15A, obtained by addition of the amino-acid sequence translated from exonIII of the PRDII-BF1 gene.

The nucleotide sequence of the invention is especially an isolated or a purified nucleotide sequence.

The invention relates especially to the use of a nucleotide sequence replying to the above definition, for the expression of a polypeptide acting as a positive transcriptional factor for a gene chosen among genes involved in the control of cellular growth, cellular proliferation, cellular differenciation or cellular apoptosis. The genes include the above cited antioncogenes.

The expression “control” of the cellular growth, cellular proliferation, cellular differenciation or cellular apoptosis means within the invention, that the used nucleotide sequence is capable of influencing, directly or indirectly, alone or as a result of a combined effect with other cellular or non cellular factors, qualitatively or quantitatively, the growth, proliferation, differenciation or apoptosis of cells.

In a particular embodiment of the invention, the deletion of the nucleotide sequence of the invention can lead to differenciation or to spontaneous apoptosis or to induced apoptosis by a genotoxic drug.

A possible assay to detect the influence of the use of the nucleotide sequence in this “control” is to compare the effect of the activity of the expression product of this nucleotide sequence on th expression of a det rmined gene, for example the p53 or the IRF1 genes, in a determined cell, to the expression of the same gene in the same cell, in the absence of expression of the nucleotide sequence of the invention.

According to a first preferred embodiment of the invention, the nucleotide sequence which is used comprises the cDNA corresponding to the 2083 nucleotide (nt) coding sequence of the transcript of the PRDII-BF1 gene; a particular coding sequence is illustrated on FIG. 14A.

According to another particular embodiment of the invention, the nucleotide sequence which is used is modified with respect to the 2.5 kb sequence or to the coding sequence represented in FIG. 14A, especially by deletion of the nucleotides corresponding to exons I and II of the PRDII-BF1 gene and a start codon is added upstream from exon III of the PRDII-BF1 gene to enable the expression of a polypeptide in a eucaryotic cell. This particular coding sequence is illustrated on FIG. 14B.

Thus in a particular embodiment of the invention, the nucleotide sequences which are used comprise the succession of exons III, V, VI, VII, VIII and IX of the PRDII-BF1 gene, said nucleotide sequence being devoid of exon IV of said PRDII-BF1 gene in the case of GAAP-1 and containing the first 45 nucleotides of exon IV in the case of GAAP-2.

According to a further embodiment of the invention, the nucleotide sequence which is used comprises the cDNA derived from the 2152 nt coding sequence of an alternative transcript of the PRDII-BF1 gene. A particular sequence of this embodiment is illustrated on FIGS. 14B and 14C.

The sequence illustrated on FIG. 14B and 14C can also be considered as a variant of the sequence illustrated on FIG. 14A. The variant represented on FIG. 14C is obtained by addition of 69 consecutive nt, i.e. addition of 24 nt in exon III and 45 nt in exon IV.

According to another particular embodiment of the invention, the nucleotide sequence which is used codes for a GAAP-1 polypeptide comprising th amino acid sequence identified under No. 2 or for a GAAP-1 polypeptide comprising the amino acid sequence identified under No. 4.

Alternatively, the nucleotide sequence used codes for a variant of GAAP-1 polypeptide, said variant being derived from GAAP-1 by insertion, deletion or substitution of one or several amino acid residues, provided it retains the property of GAAP-1 to bind an IPCS sequence and to act as a transcriptional factor for the for the transcription of a nucleotide sequence placed under the control of said IPCS sequence and present in a eucaryotic cell.

GAAP-2 can be considered as a variant of GAAP-1, by addition of 23 specific amino-acid residues translated from exons III and IV having additional nucleotide residues as a result of alternative splicing. The additional 23 amino-acid residues form a polypeptide having the following sequence with respect to the sequence of FIG. 15A:

MGQKFQKKKSYRLVLKELRNPLK.

The initiation of translation of GAAP-2 is under the control of the same ATG as that operating for GAAP-1.

The invention also encompasses the use of variants of said nucleotide sequence, especially shorter sequences, which are defined by their capacity to hybridise under stringent conditions with the DNA sequence identified under No. 1 Stringent conditions according to the invention require an hybridisation temperature of 60° C. and 3 washing steps in NaCl 40 mM SDS 0.5%.

The hybridized transcripts are then identified and selected for instance by Northern blot, or electrophoretic migration.

For the purpose of the invention, the nucleotide sequence which is used may be placed under the control of a promoter sequence selected among constitutive or inducible promoters in order to enable expression of a polypeptide. The selection of the regulation elements and especially of the promoter is dependant upon the final purpose of the expression sought. The promoters are those usually used by the skilled person in the art and may be for xample selected among the following: CMV, 5V40, β-actin, eF1α.

A particularly preferred use of a nucleotide sequence according to the above definitions is characterised by the fact that the eucaryotic cells transformed by insertion of said nucleotide sequence are malignant cells.

A “malignant cell” according to the invention is a cell whose growth, differenciation or proliferation if altered as a result of genotoxic damage, whatever their origin including as a result of ionising, radiation, genotoxic drug, viral oncogene etc.

In another particular use, the eucaryotic cells transformed by insertion of one of the above nucleotide sequences are cells of a developed tumor.

In another preferred embodiment of the invention, the use of a nucleotide sequence according to the invention enables the control of cell apoptosis.

The effect of controlling cell growth, cell differenciation, cell proliferation or cell apoptosis can be achieved in accordance to the invention through the positive regulation of transcriptional activity of a gene selected among p53, IRF1, Rb, p21 (WAF1), p27, wt1, bax, TNF receptor and FAS, or advantageously of several genes present in the treated cells, thus possibly enabling an increased efficiency.

As a result of the presence of the IPCS sequence in the promoter regions of several genes involved in the control of cell growth, cell differenciation, cell proliferation or cell apoptosis, the invention provides a way to broadly interact with this function of control of multiple genes, through a common pathway and with possibly one type of compound. The invention thus provides means which can be more efficient for the treatment of alteration of the above control.

The invention also relates to a nucleotide sequence comprising the DNA sequence identified under No. 1, or under No. 3, said nucleotide sequence being devoid of the sequence forming exon IV in the PRDII-BF1 gene.

In a particular embodiment of the invention, this nucleotide sequence consists of the DNA sequence identified under No. 1 or under No. 3 (FIG. 14A or FIG. 14B).

The invention also relates to a nucleotide sequence comprising the DNA sequence identified under No. 5, said sequence comprising 69 nucleotides in addition to that of Sequence ID No. 1.

In a particular embodiment of the invention, this nucleotide sequence consists of the DNA sequence identified under No. 5 (FIG. 14C).

This DNA sequence can be denoted cDNA sequence since it may have been isolated from a cDNA library. By the expression “cDNA” the invention not only encompasses the full-length DNA sequence which is obtained by reverse transcription of the messenger RNA. It also encompasses DNA sequences which comprise or consist of the coding sequence corresponding to an Open Reading Frame of the transcript of the gene, or any coding sequence which is sufficient when used in appropriate conditions, to enable the expression of a polypeptide having the properties defined in the present invention.

The coding sequence of the invention may thus be placed under the control of an heterologous promoter for the expression of the polypeptide, and if appropriate under the control of additional or alternative regulatory sequences.

The invention therefore encompasses said coding sequence included in a recombinant nucleotide sequence comprising regulatory sequence for cloning and/or expression in a host cell or in a target cell.

Particular nucleotide sequences derived from the above defined nucleotide sequences are those selected from the group consisting of:
(i) a fragment of the DNA sequence identified under No. 1 (FIG. 14A), or a fragment of the DNA sequence identified under No. 3 (FIG. 14B) which can be used as a specific probe to detect the presence of said DNA sequence identified under No. 1, No. 3, or No. 5 or a mutated sequence thereof,
(ii) a nucleotide sequence encoding a polypeptide having the amino-acid sequence identified under No. 2, or a nucleotide sequence encoding a polypeptide having the amino-acid sequence identified under No. 4 or a nucleotide sequence encoding a polypeptide having the amino-acid sequence identified under No. 6,
(iii) a nucleotide sequence derived from sequence defined under (i) or (ii) wherein said sequence is modified, especially by deletion, addition or substitution of one or more nucleotides providing that the resulting nucleotide sequence encodes a polypeptide capable of binding a nucleotide sequence designated IPCS which comprises the DNA sequence AAATGRYKKC, and is capable when used in appropriate conditions, of expressing in a determined eucaryotic cell, a polypeptide interacting with the nucleotide sequence designated IPCS and acting as a positive transcriptional factor for the transcription of a nucleotide sequence placed under the control of said IPCS sequence and present in said eucaryotic cell.

A preferred nucleotide sequence is that of GAAP-1 contained on plasmid pGAAP-1 deposited at the ECACC (Great Britain) under n° 01052921 on May 29, 2001.

The invention however does not relate to the PRDII coding sequence, as such, being the sequence designated PRDII on FIG. 14. It does not relate also to the encoded sequence designated PRDII which appears on FIG. 15.

PRDII coding sequence and amino acid sequence are different from GAAP-1 sequences at positions marked up by arrows in FIGS. 14 and 15.

The invention also relates to a recombinant polypeptide, being the product of the expression in a eucaryotic cell, of a nucleotide sequence coding for a polypeptide capable of interacting with the nucleotide sequence designated IPCS and capable of acting as a positive transcriptional factor for the transcription of a nucleotide sequence placed under the control of said IPCS sequence and present in said eucaryotic cell.

In a preferred embodiment, the nucleotide sequence placed under the control of said IPCS sequence is a gene involved in the control of cell growth, cell differenciation, cell proliferation or cell apoptosis.

According to another definition of the invention, the recombinant polypeptide of the invention is the product of the expression in a eucaryotic cell, of a nucleotide sequence as defined above.

Preferred recombinant polypeptides have the amino-acid sequence of FIG. 15A, 15B or of FIG. 15C.

According to a particular embodiment of the invention, the recombinant polypeptide encompassed within the above definitions is further recognised by its capacity to regulate the transcriptional activity of a gene selected among p53 and IRF1 when it is expressed in a eucaryotic cell constitutively expressing said gene.

In order to assay the capacity of such a polypeptide to achieve regulation of transcriptional activity of genes, either of pre-determined or of unknown genes, various cell lines can be used, including U937 (ATCC CRL 1593), K562 (ATCC CL 243), SK—N—SH (HTB 11), MCF7 (HTB 22), or KG-1 (CL 246). These cells are transfected with a nucleotide sequence of the invention and their binding capacity to the IPCS sequence of the p53 or IRF1 genes expressed in the tested cell line is detected and subsequent transcriptional activity is assayed.

Various methods are described in the art for the detection of the binding capacity of a compound acting as a trans-acting factor. Methods for identifying DNA-protein complexes are for example those involving analysing the product by gel retardation assays described in “Molecular Cloning—A laboratory Manual” (Sambrook and Russell, Cold Spring Harbor Laboratory Press, 2001). Methods for the analysis of transcripts of genes are also well known in the art. Such methods include for example analysing primary transcripts by transcriptional run-on as described in “Molecular Cloning—A laboratory Manual” above cited. Oth r methods also described in “Molecular Cloning—A laboratory Manual” include having recourse to reporter genes as markers of the activity of regulatory elements, and as example using CAT (chloramphenicol acetyltransferase), luciferase, or β-galactosidase genes, EGFP (Enhanced Green Fluorescent Protein).

The recombinant polypeptide according to the invention can also be defined as a polypeptide having a theoritical molecular weight of 74.7 kDa.

The molecular weight of GAAP-1 expressed in vivo has not been precisely determined since it is expressed as a fusion protein after transfection of Hela cells with a recombined nucleotide sequence GAAP-1/EGFP. The transfected cells have been fractionated by electrophoresis on SDS-PAGE gel and revealed after blotting by an antibody directed against EGFP. The molecular weight of the fusion protein is between 98 and 64 kDa.

In a particular embodiment, the recombinant polypeptide, being the expression product in a eucaryotic cell is expressed in a mammalian cells, especially in a human cell, and is modified as a result of post translational modifications occurring in the eucaryotic cells, including proteolytic cleavage and/or phosphorylation, ubiquitinization, sumoylation.

Phosphorylation can be detected on the expressed recombinant product, by using antibodies directed against the potentially phosphorylated amino acid residues, i.e. tyrosine, serine and threonine.

A variant recombinant polypeptide according to the invention can be obtained as a result of the mutation of the phosphorylated amino acid residues, provided the mutated polypeptide retains the binding and transcriptional activity of the original one.

A preferred recombinant polypeptide according to the invention comprises the amino acid sequence identified under No. 2 or under No. 4 or under No. 6 (FIGS. 15A, 15B and 15C).

Another particular polypeptide according to the inv ntion has an aminoacid sequence shorter, which is comprised within the the amino acid sequence identified under No. 2, or under No. 4, or under No. 6 or a variant thereof replying to the definitions given above, provided the obtained polypeptide is capable of interacting with the nucleotide sequence designated IPCS to act as a positive transcriptional factor for the transcription of a nucleotide sequence placed under the control of said IPCS sequence and, present in a eucaryotic cell.

The invention also relates to the polypeptide having the following sequence: KSYRLVLKELRNPLKR. This polypeptide can be used as antigen for the preparation of an antibody which would recognize GAAP-2 and would not cross react with PRDII-BF1 and GAAP-1.

The invention further concerns recombinant eucaryotic cells which are recombined by insertion of a nucleotide sequence according to the definitions provided hereabove.

In a particular embodiment, the recombinant eucaryotic cell expresses the polypeptide encoded by the inserted nucleotide sequence, which polypeptide has the above defined properties.

The insertion of the nucleotide sequence of the invention can be achieved by transfection having recourse to known methods such as electroporation or other methods including those described in “Molecular Cloning—A Laboratory Manual” (Cold Spring Harbour Press—2001).

Particular recombinant eucaryotic cells of interest are selected among cells normally expressing genes involved in the control of cell growth, cell differenciation, cell proliferation or cell apoptosis.

Such cells include, but are not limited to, cell lines used for the study of the properties of the expressed products, such as U937, K562, SK—N—SH, TF1, MCF7, or KG-1 cell lines.

In a particular embodiment of the invention, especially in relation with the proposed use of the described means for a therap utic, diagnostic or prognostic of alteration of the regulation of the transcription of genes involved in the control of cell growth, cell differenciation, cell proliferation or cell apoptosis, recombinant eucaryotic cells are cells obtained from a patient, are especially malignant cells or cells constituting a potential candidate for the occurrence of said alterations.

The recombinant eucaryotic cell according to the invention can also be defined as a cell which is recombined with a nucleotide sequence encoding a polypeptide capable of binding a nucleotide sequence designated IPCS and comprising the DNA sequence AAATGNNNNC, for the expression in a determined eucaryotic cell of a polypeptide capable of interacting with the nucleotide sequence designated IPCS and capable of acting as a positive transcriptional factor for the transcription of a nucleotide sequence placed under the control of said IPCS sequence and present in said eucaryotic cell.

In a particular recombinant eucaryotic cell according to the invention, the polypeptide comprises the amino acid sequence identified under No. 2 or under No. 4 or under No. 6 (FIGS. 15A, 15B and 15C).

Particular recombinant eucaryotic cells useful for the invention are selected among stem cells or differenciated cells including U937, K562, SK—N—SH, MCF7, KGI, TF1.

The invention also concerns recombinant polypeptides which are fusion polypeptides including those wherein the polypeptide of the invention is fused with a reporter sequence, for instance fused with EGFP or EGFP . . . FcIgG1.

The invention is further directed to a recombinant vector which is recombined with an insert consisting of a nucleotide sequence of the invention.

Particular recombinant vectors are expression vectors, suitable for expression of said insert, in a eucaryotic cell, especially in cells selected among the above cited cells.

In a particular embodiment of the invention, the recombinant vector is suitable for transient expression or for controlled expression of said insert. Transient expression may be required for therapeutic purposes in order to avoid undesirable ffects of the expression of the inserted construct in cells. Controlled expression includes the possibility of obtaining induced expression, or more generally the possibility to control the level of the expression of the inserted construct.

In order to obtain said controlled expression, said insert can be placed under the control of an inducible promoter, advantageously a promoter regulated by a physiologically acceptable compound. Inducible promoters are for example IPTG-inducible promoters such as the trplac (tac) promoter, the trp-lac (trc) promoter, the lac promoter, which are used in pUC, pSKpTZ, pGEM, pBluescritp vectors.

In another particular embodiment of the invention, in the recombinant vector the transcription of said insert is placed under the control of an exogenous transactivating system. Particular artificial systems for the control of the transcription encompass the tetracycline modulated system for example with the use of a pTRE vector (Clontech), the rapamycin inducible system Ponasteron Inducible System.

The recombinant vector according to the invention is advantageously designed in such a way that it is suitable for gene therapy.

For this purpose the recombinant vector can be selected among viral, retroviral, lentiviral, poxviral, adenoviral, AAV vectors. The known requirements and advantages for the design of safe and efficient vectors should be taken into consideration in preparing these vectors. A review of the techniques in this field is available in La Therapie génique (Editions TEC & DOC, 2001).

The invention also concerns a composition suitable for therapeutic use, which comprises a nucleotide sequence as defined above, or a recombinant cell according to the invention, or a recombinant polypeptide of the invention.

Such a composition may be used in a particular embodiment, in combination with a therapeutic agent selected among antiviral agents and anticancer agents. The combination means that the composition of the invention may be associated with the anticancer agent in the protocol. The administration of these associated compounds can be done under the form of a single composition, or alternatively, the composition of the invention and the anticancer agent can be administered as separate compositions, including the possibility for a simultaneous concomitant or sequential administration in time.

The anticancer agent can be an immuotherapeutic agent, a chemotherapeutic agent or can be radiotherapy. Appropriate agents include 5FU, cisplatine, etoposide, cytokines.

The combined use propose may enable to reduce the level of the efficient doses of the anticancer agent used.

The composition of the invention can be included in a therapeutic protocol for the treatment of malignant cells or for the treatment of tumors.

The expression “treatment” within the present invention encompasses the effect produced on the control of cell growth, cell differenciation, cell proliferation, tumorigenicity, cell apoptosis which in order to inhibit, circumvent or even reverse the development of the malignant state, including the capacity of preventing or inhibiting the growth and/or spreading of a tumor.

Cell differenciation can be assayed, for instance for erythroleucemic cells (K562, UT7) by their capacity to produce haemogloblin.

Cell proliferation can be evaluated by measurement of the reduction of MTT or a similar assay. The cells can be injected to nude mice in order to determine their tumorigenic capacity.

Apoptosis can be assayed by the “TUNEL” method or by labeling with annexin V.

The invention thus relates to the use of a nucleotide sequence, or of a recombinant cell, or of a recombinant polypeptide, for the preparation of a biological tool for screening compounds capable of interacting with an IPCS sequence present in the promoter sequence of a gene involved in the control of cellular growth, cellular proliferation, cellular differenciation or cellular apoptosis, and capable of regulating the transcription of a nucleotide sequence placed under the control of said IPCS sequence.

The inventors have defined a novel approach for the regulation of genes involved in the control of the evolution of cells toward a malignant cells which approach offers means interacting with a potentially broad spectrum of target genes to control their regulation.

The invention further discloses novel means useful for the detection of an alteration of the control of the regulation pathway of the transcription of genes involved in the control of cell growth, cell differenciation, cell proliferation, cell apoptosis. The defined means are based on the detection of an abnormal BRDII-BFI gene in cells, either as a result of a mutation affecting the transcription of the gene normally producing the spliced form of messenger RNA corresponding to the nucleotide sequence of the invention, or affecting the proper translation of said transcript. The presence of an abnormal gene can be correlated to a potential deficient control of genes containing in their promoter region a sequence of the IPCS type and as a consequence, to the possibility for a cell to develop as a malignant cell or to the possibility for a malignant cell to be treated according to the invention.

The invention therefore relates to a process for the in vitro detection of a deficient BRDII-BFI gene comprising the steps of:
contacting a probe constituted from the nucleotide sequence identified under No. 1 or under No. 3, or under No. 5, or a fragment thereof comprising the zinc finger binding domains corresponding to the domains localised within exon VI of the BRDII-gene, with the DNA of a cell normally constitutively expressing said gene, in stringent conditions,
detecting the hybridisation product between said probe and said cell DNA. One appropriate probe can be the nucleotide sequence spanning exon 5 to exon 9, i.e., nucleotides 6396 to 9020 of the sequence identified under No. 1.

The invention also provides a process for the in vitro detection of deficient transcriptional activity of genes involved in the control of cell growth, cell differenciation, cell proliferation or cell apoptosis, comprising the step of detecting deficient production of the transcript of said gene which would normally encode a polypeptide capable of binding an IPCS sequence and as a result would positively regulate the transcription of a nucleotide sequence placed under the control of said IPCS sequence.

According to another embodiment of the invention, a process is defined for the in vitro detection of a prognostic of transformation of cells toward a malignant state, which comprises the step of detecting a mutation in the PRDII-BF1 gene normally expressed in said cells, or detecting a mutation in the transcript obtained by splicing of said gene, which mutation would result in lack of expression or in an abnormal expression of polypeptide expression product of said PRDII-BF1 gene capable of binding to an IPCS sequence.

The invention also concerns a process for the screening of compounds capable of regulating the transcriptional activity of genes containing an IPCS sequence in their promoter region, said genes being active in the control of cell growth, cell differenciation, cell proliferation, or cell apoptosis, said process comprising the steps of:
contacting the assayed compounds with the DNA of a cell expressing genes containing an IPCS sequence in their promoter region,
detecting a DNA-compound complex formation and assaying its transcriptional activity on said gene containing the IPCS sequence.

The DNA of the cell can be used under the form of a nuclear extract of the cell.

The invention also relates to the use of the described polypeptide for the screening of molecules, especially small molecules having interest for administration to patients, wherein said molecules are selected either for the agonist properties with respect to GAAP-1, or to GAAP-2, or for their antagonist properties with respect to GAAP-1 or to GAAP-2.

Agonists of GAAP-1 or of GAAP-2 may be suitable for the preparation of anti-cancer agents. Antagonists of GAAP-1 or of GAAP-2 may be suitable for the preparation of compositions having interest in the treatment of inflammatory or autoimmune diseases.

Numerous methods are available for the screening of such compounds especially for the detection of the DNA-compound formation. This step can be carried out using gel retardation assay, supershift assay, competition assay. Techniques involving immunoblotting or immunoprecipitation assays may also be appropriate.

The identification of compounds capable of inhibiting or stabilising the formation of the IPCS/GAAP-1 complex, can be carried out by a gel shift technique.

The identification of compounds inhibiting or favoring the recruitment of other proteins capable of acting as partner with GAAP-1 or with GAAP-2 can be carried out by co-immunoprecipitation.

Identification of compounds capable of inducing translocation between cytoplasm and nucleus can make use of immunohistochemistry.

Compounds capable of inducing postranslational modifications of GAAP-1 or of GAAP-2 such as phosphorylation, ubiquitinization, sumoylation, acetylation can be identified by Western blot.

The invention will be further illustrated by the following examples and figures.

### LEGEND OF THE FIGURES

FIG. 1A Schematic representation of the reporter construct used to clone IPCS-BF1. The IPCS from p53 or IRF1 gene or the mutated IPCS is cloned upstream His3 minimum promoter. FIG. 1B Confirmation of the IPCS-BF1 binding specificity. After the transformation by IPCS-BF1/Ga14AD (IPCS-BF1/Ga14) fusion protein or empty expression vector (control) the IPCS yeast reporter strain are spread on selective medium lacking histidine. As each reporter strain possess its own leaky expression of HIS3, the 3AT was adjusted at for IPCS-p53, IPCS-IRF1 and IPCSM-P53 at 15, 25 and 40 mM respectively. FIG. 2A—Schematic representation of the exon composition of PRDII-BF1 and IPCS-BF1. This data are deduced from the work of gaynor (91) and Xu (Xu 99) The nucleotide positions are as in Fan & Maniatis (1990).

FIG. 3—Deduced amino acid sequence of IPCS-BF1. The putative NLS, the two zinc fingers domain and the PEST domain are framed.

FIG. 4a)—Plasmid constructs encoding an EGFP/GAAP-1 fusion protein for transient expression.

FIG. 4b)—Sub-cellular localisation of EGFP/GAAP-1 variants. HuH7 cells were transiently transfected with the expression vector indicated. After 24 h, the nuclei were stained with Hoechst 33342 and examined by confocal microscopy.

FIG. 5a)—EMSA using the IPCS-p53 probe and in vitro translated GAAP-1 (lane1), GAAP-1 mtZ1 (lane 3), GAAP-1 mt Z2 (lane 5), or reticulocyte lysat with the empty expression vector (lane 7). Even lanes: 50 fold molar excess of IPCS-p53 oligonucleotide was added to the reaction of the previous lane. The arrow indicates the specific complex.

FIG. 5b) The same as in B except that the IPCS-p53 probe was replaced by the NF-κB oligonucleotide.

FIG. 5c) Amino acid sequence of the two zinc finger domains. The residues which are substituted in GAAP-1 mtZ1 or in GAAP-1 mtZ2 are underlined.

FIG. 6—Determination of post-translational modifications of GAAP-1. Western blot analysis of 20 μg of cellular extract from HeLa cells stably transfected with either an EGFP, EGFP/GMP-1, GAAP-1/EGFP, EGFP/GAAP-1 del PEST expression vector, and probed with anti-EGFP antibodies.

FIG. 7—Analysis of the effect of intramolecular cleavage on the DNA binding properties of GMP-1, by EMSAs with nuclear extracts of K562 cell stably transfected with EGFP/GAAP-1 expression vector.

FIG. 8—Relative activity of the wild type and mutated p53 and TRF1 promoters, IPCS-SV40 chimeric promoter, and the HIV-LTR in a cotransfection assay. U937 cells were tansiently co-transfected with 3 μg of the reporter plasmids and 7 μg of the expression vectors indicated. The pcDNA3.1 vector was used as a control. The pRL-SV40 plasmid was used as an internal control for the normalisation of the transfection efficiency. The transfected cells were harvested 8 h after transfection and assayed for luciferase activity.

FIG. 9—U 937 and K562 cells were stably transfected with GAAP-1 or EGFP expression vectors and selected using G418. in semi-solid methyl-cellulose medium. The number of clones was determined at 21 days.

FIG. 10—Involvement of GAAP-1 in apoptosis. Spontaneous apoptosis of U937 cells stably transfected with EGFP or EGFP/GAAP-1 expression vectors was assayed by annexin V-PE/7-AAD double labelling.

FIG. 11—Western blot analysis of 20 μg of cellular extract from U937 cells stably transfected with either an EGFP or EGFP/GAAP-1 expression vector, and probed with anti-p53 or anti-IRF1 antibodies.

FIG. 12—U937 cells stably transfected with either the EGFP or EGFP/GAAP-1 fusion protein expression vectors were treated or not with 1 mM 5FU. Apoptosis was analysed by the TUNEL assay 72 hours after treatment.

FIG. 13—Influence of expression of GAAP-1 on spontaneous or induced differentiation by A2ra-c or Hemin of K562 erythroid cells.

FIGS. 14A and 15A—GAAP-1 and PRDII sequences FIGS. 14B and 15B—variants of GAAP-1 nucleotide and amino-acid s quences.

FIGS. 14C and 15C—GAAP-2 sequences

FIG. 16—Map of plasmid pGAAP1 (ECACC 01052921)

### EXAMPLES

The constitutive expression of the tumour suppressor genes p53 and IRF1 (interferon regulatory factor 1) is required for the maintenance of the cellular growth control during genotoxic damage or aberrant proliferation. Following DNA damage, the activity of p53 is enhanced and its level rises considerably, mainly by protein stabilisation. p53 exerts its effect by transcriptional induction of genes involved in cell growth arrest which allows DNA repair or eventually apoptosis (Vogelstein 00) Mutations in the coding region of the p53 gene, which have been observed in more than 50% of human cancers (Soussi 00), inactivation of the protein by the association with the viral oncoproteins E1A or E6, or deregulation of factors implicated in its turnover are the most frequent causes for an altered response to DNA damage (Vogelstein 00). However, the absent or weak expression of the p53 gene has also been observed in various tumoural cell lines (Raman 00) where this gene is apparently fully functional. This suggests that transcriptional deregulation of the p53 gene can be considered among the mechanisms involved in the loss of p53 function.

The transcription factor IRF1 has been originally identified as a positive regulator of the interferon system (Miyamoto Fujita). Accumulating evidence also suggests that IRF-1 controls tumour susceptibility. Thus, the transformed phenotypes of c-myc or fos-B expressing cells can be suppressed by ectopic expression of IRF-1 (Tanaka 94). In addition, while c-HA-ras is unable to transform primary mouse fibroblasts (MEFs), it does transform fibroblasts originating from knockout mice for IRF1 (Tanaka 94a). IRF1 is necessary for genotoxic stress cell cycle arrest (Tanaka 96) and is responsible for the activation of at least two genes, p21waf1 and p27kip1, which play a central role in cell cycle control (Tanaka 1996 Coccia99, Moro 00). Furthermore, IRF1, like p53, is involved in the induction of the apoptotic genes caspases (Furukawa 99) and Bax. IRF-1 is also implicated in the FAS apoptotic pathway. Indeed, the FasL promoter possess a specific binding site for IRF-1 which directly controls the inducibility of FasL by TCR (Wa 00). Alterations of the IRF-1 gene are also associated with various diseases. Chromosomal deletion or inactivation of one of the two alleles of the IRF1 gene are commonly associated with human leukemic or preleukemic syndromes (Taniguchi 1995). Loss of functional IRF1 mRNA by skipping specific exons has been reported in 20% of patients with myelodysplastic syndrome (Harada 94). Finally, loss of heterozigosity at the IRF1 locus is frequently observed in patients with gastric or esophagic cancers (Ogasawara 96 Tamura 96).

The expression of the p53 gene is inducible by a number of different stimuli. Several different regulatory elements are present within the p53 promoter, such as a genotoxic stress response sequence, extending from positions −70 to −40 (24), a binding site for c-Myc-Max heterodimers (25), two overlapping binding sites for NF1 and YY1 from positions −227 to −194, implicated in the constitutive expression of the p53 gene, (27) and a site for HOXA5 at position −204/−201. HOXA5 is a positive regulator of p53 transcription in the normal breast epithelium and lack of its expression may be the primary cause for loss of p53 expression in human breast cancer cells (Raman 00).

The expression of the IRF1 gene is strongly induced by both IFN-alpha/beta and IFN-gamma. This property is due to the presence of a regulatory element called IR (inverse repeat) within the IRF1 promoter (28), which also contains several putative Sp1 sites, probably involved in the basal transcriptional activity of this gene (21, 28).

The p53 and IRF1 promoters also contain NF-κB sites (28, 30), which confer upon each gene inducibility by a variety of different agents such as oxidative stress, IL-1β, and TNF-α. Other common features of the two genes are the constitutive and ubiquitous expression in the quasi-totality of untransformed tissues (22) and the absence of TATA boxes (28, 31, 32).

The present invention describes the cloning of the GAAP-1 cDNA and presents data on the structure and function of this factor. The structure of GAAP-1 has been correlated to some of its features such as sub-cellular localisation, DNA binding and transactivation activity.

Experimental Procedure

Cloning of GAAP-1

S. cerevisiae YM4271 and the reporter vector pHISi were obtained from Clontech. The reporter constructs were generated by inserting one copy of the double stranded oligonucleotides 5′AAAATGATTTCCAC3′, ′-AAAACGATTTCCAC-3′, 5′-GAAATGACGGCACG3′, corresponding respectively to the IPCS-p53, IPCS-p53M and IPCS-IRF1 sequences previously identified (Lallemand 97), into the EcoRI and XbaI sites of the pHISi. These plasmids were linearised and used to transform YM2471 competent cells. The yeast reporter strains were maintained by selection on synthetic dextrose medium lacking histidine. As each yeast reporter strain possess its own leaky expression of HIS3, 20 mM, 30 mM and 45 mM of 3-AT (3-amino-1,2,4-triazole) were added to the medium to suppress the background growth of respectively IPCS-p53, IPCS-IRF1 and IPCS-p53m yeast reporter strains. Screening of the human leukocyte cDNA library (Clontech) encoding proteins fused with the GAL4 activation was performed by the lithium acetate method (Gietz 92) in a yeast strain carrying the HIS3 reporter gene under the control of one IPCS-p53 sequence. The transformed yeast cells were plated under selective conditions in synthetic medium containing 20 mM 3AT and lacking histidine and leucine. In order to confirm the transactivation properties of positive clones, plasmids were recovered from these clones by a rapid isolation procedure (Kaiser & Auer 1993), and used to transform E. coli. A mini-preparation (Wizard, Promega) of these plasmids were then used to transform yeast strains carrying the HIS3 reporter gene under the control of the IPCS-p53, IPCS-IRF1 or IPCS-p53M sequences. Only plasmids which can confer auxotrophy to histidine to strains containing IPCS-p53/HIS3 and IPCS-IRF1/HIS3 and non IPCS-p53M were retained.

Cell Lines and Drugs

U937 cells (ATCC CRL 1593), derived from a human histiocytic lymphoma and K562 (ATCC CL 243), derived from a human myeloid leukemia were cultivated in RPMI 1640 medium supplemented with 10% fetal calf serum (Life Technologies, Inc.) Hela cells (ATCC CCL-2) were cultivated in DMEM medium supplemented with 10% fetal calf serum. 5 fluorouracil, cytosine β-D-Arabinofuranoside and hemin were purchased from Sigma-Aldrich.

Plasmids

The GAAP-1 expression vector was constructed by cloning the PCR product (nt 6396 to 8601) from human leukocyte cDNA library (Clontech Laboratories) in the pcDNA3.1 expression vector (Invitrogen) using the following primers: 5′-AGCATGGCATTAGGTAATCAAAAGTCCACAG-3′ 5′CCATCAGGTTGCTATCACAAGC-3′. The plasmid expressing the EGFP/GAAP-1 fusion protein was obtained by fusion PCR (Higuchi, R., In PCR protocols, a guide to methods and applications (Eds Innis, M. A., Gelfand,) using the following primer couples (5′-ACCATGGTGAGCAAGGGCGA-3′, 5′-GATTACCTAATGCTCTCTTGTACAGCTCG-3′) and (5′-GGACGAGCTGTACAAGATGGGGCAGAAGTTTCAAAA-3′, 5′ CCATCAGGTTGCTATCACAAGC-3′) to amplify respectively the EGFP and GAAP-1 coding region. The EGFP/GAAP-1 del NLS, the GAAP-1 mtZ1 and the GMP-1 mtZ2 were obtained by overlapping PCR (Higuchi above) using the following overlapping primer couples (5′-CCTTAATCAAAAGTGAAGATGGAGGATATAAGTCA-3′, 5′-ATCCTCCATCTTCACTTTTGATTAAGGAATT-3′), (5′-GCAGAAGAAGCTGGAATACGTTGTAAGMAC-3′, 5′-AGCTTCTTCTGCAATGTATTTCCTCTTCC-3′) and (5′-GCAATGAAGTCCAAGGCAGGAAGCAAGAAATGTGTGGA-′, 5′-TCCTGCCTTGGACTTCATTGCTTTTGTCAGATTTCC-3′) respecitvely. All the coding regions were cloned in the expression vector pcDNA3.1. (Invitrogen). The integrity of the constructs was verified by sequencing. The pRL-SV40 was purchased from Promega (Promega).

Transfection

Transfection was performed with 10 μg DNA per 10⁶ cells/0.8 ml medium by electroporation as described in Lallemand 97. Cells were transfected with DNA using Superfect (Qiagen) according the manufacturer's instructions. The pRL-SV40 (Promega) was used as an internal control for the normalization of the transfection efficiency. The transfected cells were harvested 24 h after transfection and assayed for luciferase activity using the Dual-luciferase Reporter assay System (Promega).

Western Blot Analysis

Ten micrograms of nuclear extract, prepared as described below, were denatured by boiling for 5 min in loading buffer (100 nM Tris, 12,1% glycerol, 2.4% SDS, 1.4% b-mercaptoethanol, 2.3% bromophenol Blue), then fractionated in 12% SDS-PAGE and transferred onto PVDF membrane filter. The immunodetection was carried out as described elsewhere (ref) with anti-GFP (Zymed), anti-p53 (pAb 240) (Santa Cruz Biotechnology Inc.) or anti-IRF1 (Santa Cruz Biotechnology Inc.) antibodies. Signals were detected with an ECL kit (Amersham).

Nuclear Extracts

Nuclear extracts were prepared using a modification of the procedure described by Osborn et al. (34). Briefly, 10⁷ cells were washed twice with phosphate-buffered saline, lysed with 20 μl of a buffer containing 10 mM HEPES, pH 7.9, 0.1% Nonidet P-40 (Fluka), −1.5 mM MgCl2, 10 mM KCl, 0.5 mMDTT, and the following protease inhibitors: 1 mM phenylmethylsulfonylfluoride, 50 μg/ml [alpha ]-phenylmethylsulfonyl fluoride, and 5 μg/ml each of leupeptin, pepstatin, aprotinin, and antipain). Samples were incubated for 15 min on ice, and the cellular lysate was vortexed briefly and centrifuged in a microcentrifuge for 10 min at 4° C. Nuclear pellets were suspended in 15 μl of extraction buffer (20 mM HEPES, pH 7.9, 25% glycerol, 420 mM NaCl, 1.5 mM MgCl2, 0.2 mM EDTA, 0.5 mM DTT, together with the same protease inhibitors as described above), and incubated for 15 min at 4° C., mixed briefly, and centrifuged in a microcentrifuge for 10 min at 4° C. The supernatant was then added to an equal volume of storage buffer (20 mM HEPES, pH 7.9, 20% glycerol, 0.2 mM EDTA, 0.5 mM DTT, together with the same protease inhibitors as described above) and then stored at −70° C.

EMSA

Synthetic double stranded oligonucleotide probes were labelled with α-³²P dCTP (Amersham Corp.) by “filling-in” with Sequenase, and then purified on Sephadex G-50. The protein-DNA binding reactions were carried out using 5 μg of nuclear extract and 0.1 fmol of probe in 20 μl of binding buffer (20 mM Tris-HCl, 25 mM KCl, 1 mM DTT, 1 mM EDTA, 0.1% Nonidet P40, 4% glycerol, 1 mg/ml bovine serum alcbumin, μg/ml poly(dI-dC)) for 20 min at 4° C. In vitro translation products were generated by 50 μl of TNT coupled reticulocyte Lysate System (Promega) and 1 μg of the appropriate plasmid. 5 μl of the in vitro product was used instead of the nuclear extract when required. The reaction mixture was then electrophoresed on a 6% non denaturing acrylamide gel. Competition experiments were carried out using a 50-fold molar excess of the unlabelled probe. The following oligonucleotides (and their complementary strands) were used in these studies:

| | |
|---|---|
| IPCS- | 5′-AGCCTGATTTCCCCGAAATGACGGCACGCAGCC-3′; |
| IRF1, | |
| IPCS-p53, | 5′-AATGCAGGATTCCTCCAAAATGATTTCCAC-3′; |
| NF-κB-MHC | 5′-GATCCTCTGGGGATTCCCCATGGA-3′. |

Detection of Apoptosis
Spectrophotometric Assay for the Hemoglobin Levels

The hemoglobin levels were determined by a slight modification of a previously described method (Ray 96). 5×10⁵ cells were washed twice with PBS, resuspended in 35 ul of passive lysis buffer (Promega). The cell lysate was centrifuged at 14000 rpm in a microfuge for 15 min a 4° C. 30 ul of supernatant was mixed with 100 ul of freshly prepared benzidine-HCl (10 mg/ml in 0.5% acetic acid) and 1 ul of 30% H₂O₂ in a 96 wells microtiter plate. After 90 s the absorbance at 604 nm was measured in a microtiter plate reader (ref).

RESULTS

Cloning and Characterisation of the GAAP-1 cDNA.

A common sequence exists in the p53 and IRF1 promoters (IPCS) which has been reported previously to bind a constitutive factor present in nuclear extracts of U937 cells and to direct the transcriptional activation of a reporter gene. This constitutive factor has been identified as a 26 kDa protein consisting in a single polypeptide chain (Lallemand 97).

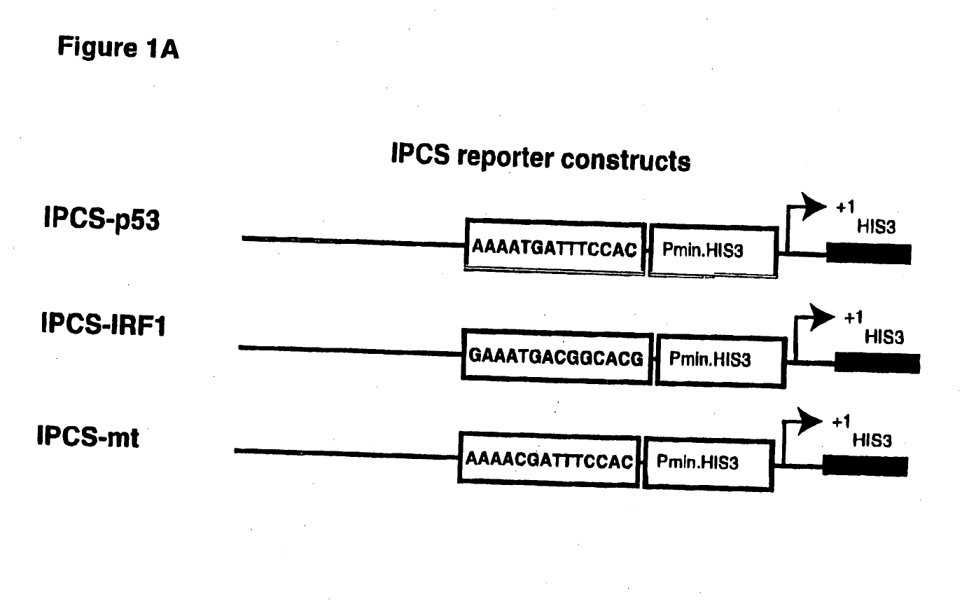

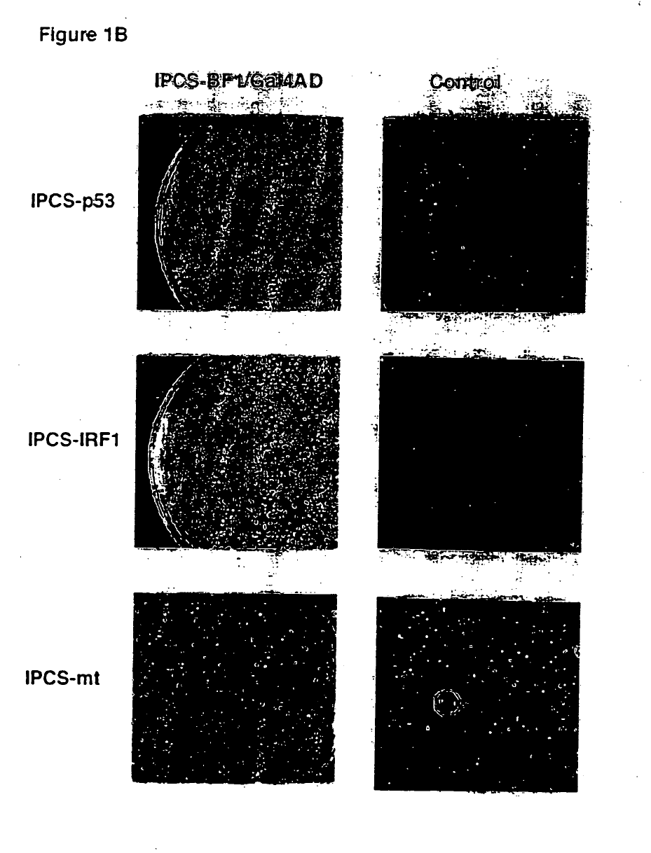

To identify complementary DNAs encoding proteins able to interact with the IPCS sequence we used a yeast one-hybrid system. A yeast tester strain, YIPCS, was established by introducing the reporter plasmid, pHISi-IPCS, into strain YM4271 (Clontech) (HisUraLeu). The pHISi-IPCS contained 14-bp of IPCS from the p53 promoter (IPCS-P53) corresponding to the sequence AAAATGATTCCAC, upstream of the minimal promoter of the HIS3 gene of the pHISI vector (Clontech laboratories). Strain YIPCS was transformed by a human leukocyte cDNA library (Clontech), in which cDNAs were fused with the GAL4 activation domain, and plated on medium lacking leucine and histidine and containing 20 mM 3-AT. From 10⁷ transformants in this manner, we isolated a 2.5 kb cDNA clone coding for a protein fused with the GAL4 activation domain. To control the specificity of the interaction of this protein with IPCS, we transformed yeast cell lines harbouring a HIS3 reporter gene under the control of different IPCS sequences (FIG. 1A) with the 2.5 kb cDNA clone. The hybrid protein was able to up-regulate the HIS3 gene controlled by the wild type IPCS from p53 and IRF1 genes, but not the one controlled by a mutated IPCS (FIG. 1B). This result suggests that the protein coded by the 2.5 kb cDNA clone possess binding properties for the IPCS sequence from the p53 promoter (nucleotides 6396 to 6488).

Sequence analysis of the 2.5 kb cDNA, called GAAP-1 cDNA, revealed 95% homology to the human PRDII-BF1 cDNA (Fan 90 etc.) in a region comprised between nt 6488 and nt 9020 (relative to the ATG of PRDII-BF1 in Fan 90) (FIG. 2A). It was previously demonstrated that a 2.5 kb mRNA could be produced from the PRDII-BF1 gene by skipping exon 4, thus it contained exon 3 directly fused to exon 5 (Gaynor 91). Except for the first 90 bp, which are not encompassed by the GAAP-1 cDNA and are substituted by the sequence coding for the GAL4 activation domain in the clone obtained from the cDNA library, GAAP-1 cDNA corresponds exactly to the transcript of 2.5 kb described by Gaynor et al.

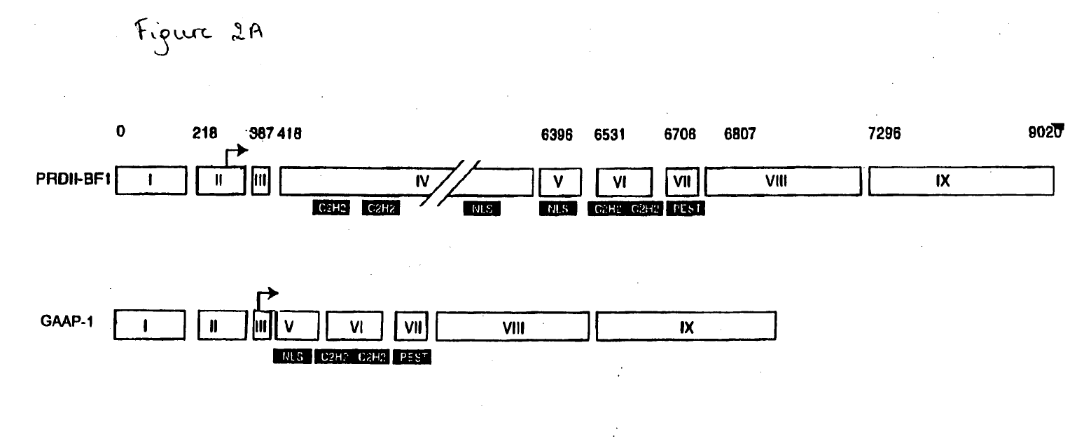

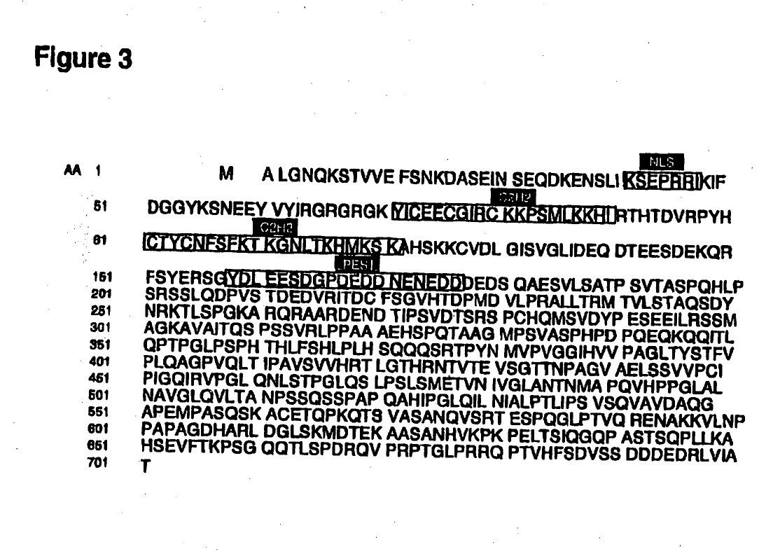

Examination of the amino acid sequence encoded by the GAAP-1 cDNA revealed the presence of three major motifs (FIG. 3): a putative NLS (PRRIKIF) distinct from the putative NLS previously identified (Fan 90 Baldwin), two C2-H2-type Zinc fingers previously characterised (Fan 90, Gaynor 91, Baldwin 90), and an acidic-amino acid rich domain similar to a PEST sequence (Rechsteiner 96) whose function is still unknown.

Sub-Cellular Localisation of GAAP-1

As GAAP-1 cDNA did not encompass the NLS already identified in PRDII-BF1 (9 kb) at 4158, Baldwin 90), we tested the activity of the NLS present in GAAP-1.

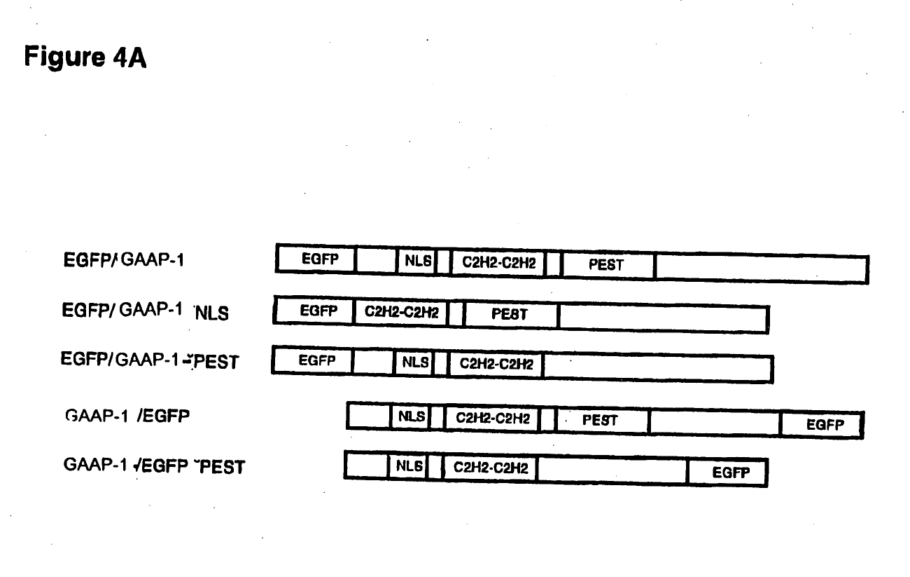

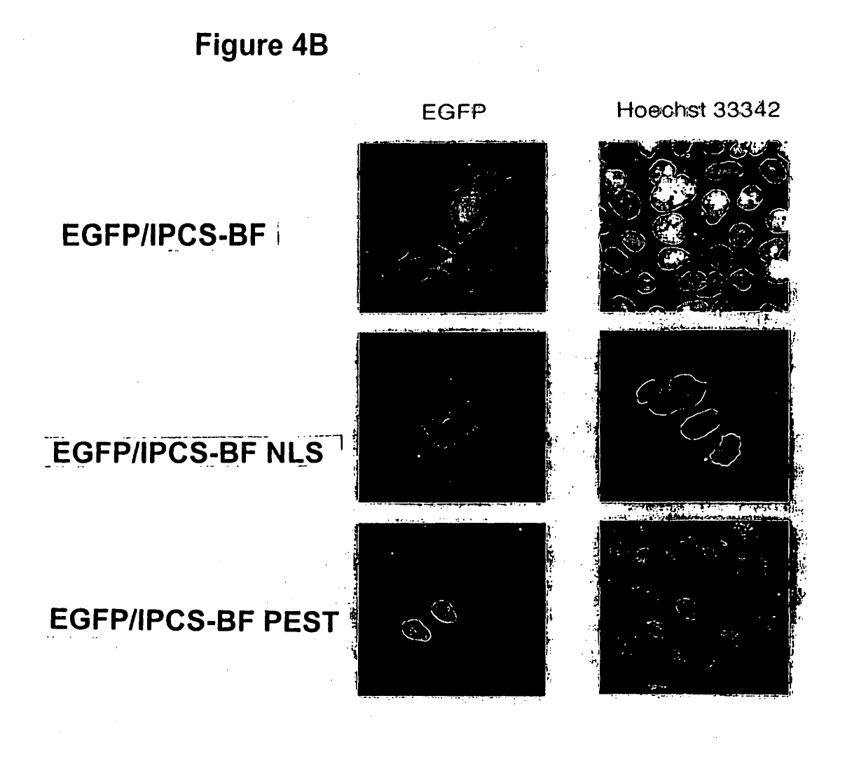

In order to determine the sub-cellular localisation of GAAP-1, we performed transient transfection assays with various plasmid constructs encoding an EGFP-GAAP-1 fusion protein (FIG. 4A). To facilitate determination of the localisation of fluorescence, we used the human hepatoma mono-layer cell line HuH7 (Bressac 90). As shown in FIG. 4B., the fluorescence of the wild type EGFP-GAAP-1 was localised both in the nucleus and cytoplasm. This distribution suggests that the activity of GAAP-1 may be regulated by the control of its compartmentalisation. The fluorescence of the NLS deleted mutant was restricted to the cytoplasm, demonstrating that the PRRIKIF motif was indeed required for the import into the nucleus. More surprisingly, transfection of the fusion protein deleted of the PEST-like sequence produced a strictly nuclear fluorescence. Similar results have also been obtained in the diploid human fibroblastic cell line MRC5 (Maziere 00) (data not shown). All these data suggest that GAAP-1 possess a least two domains implicated in its translocation: an NLS, which is distinct from the previously defined NLS of PRDII-BF1, and a PEST-like sequence which may allow the export of the protein from the nucleus, in order to be degraded into the cytoplasm.

DNA Binding Activity of GAAP-1

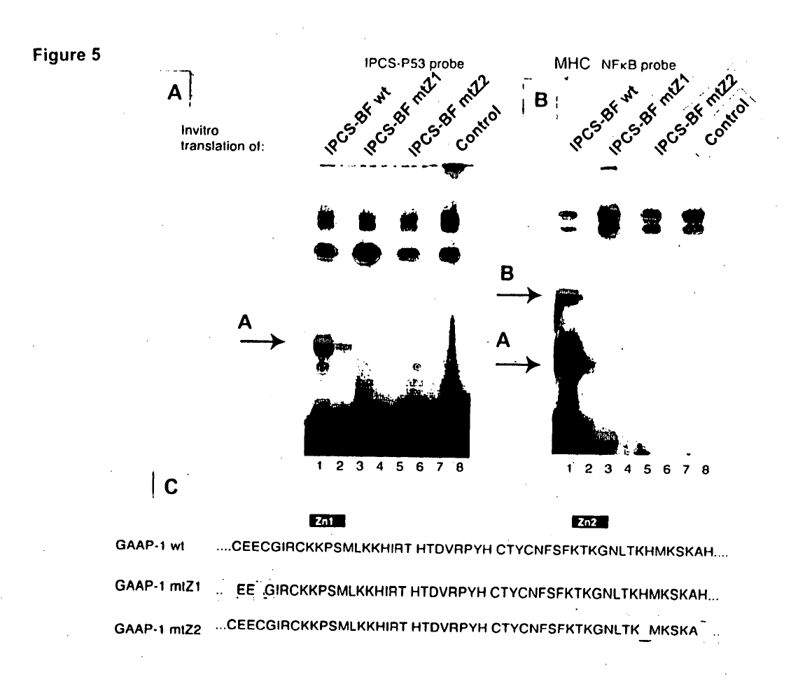

PRDII-BF1 was previously described as a protein binding to an NF-κB site whose ability to recognise the DNA is determined by two pairs of zinc fingers, one of them present in GAAP-1 (Fan 90, Gaynor, Baldwin). However, the cloning of GAAP-1 was realised using a sequence unrelated to the NF-κB sequence, suggesting that this factor was able to bind to at least two non homologous DNA sequences. To confirm the binding specificity of the cloned GAAP-1 for the IPCS target sequence, in vitro translated GAAP-1 was incubated with labelled IPCS or NF-κB oligonucleotide and analysed by Electro Mobility Shift Assay (EMSA). As shown in FIG. 5A, GAAP-1 was able to form one complex with IPCS (FIG. 5A, lane 1) and two complexes with the NF-κB oligonucleotide (FIG. 5, lane 1), demonstrating the double specificity of GAAP-1 binding. Furthermore, the affinity of in vitro translated GAAP-1 was considerably stronger for the NF-κB probe than for the IPCS-P53 probe. The formation of the slower migrating complex observed on the NF-κB sequence (FIG. 5B, lane 1) suggested an homodimerisation of GAAP-1. As GAAP-1 possess two zinc fingers, we hypothesised that each finger was responsible for the binding of each type of sequence. We constructed two GAAP-1 mutants, each containing either of the zinc fingers, the other being destroyed by substitution of the cysteine or histidine, necessary for the tertiary structure of a finger, by an arginine (FIG. 5C). In EMSA, the two in vitro translated mutants could not form any complex with either IPCS-p53 (FIG. 5A, lanes 3,5) ) or NF-κB (FIG. 5B, lane 3,5) probe. Thus, both zinc fingers were required for the binding of GAAP-1 to both IPCS and NF-κB sequences.

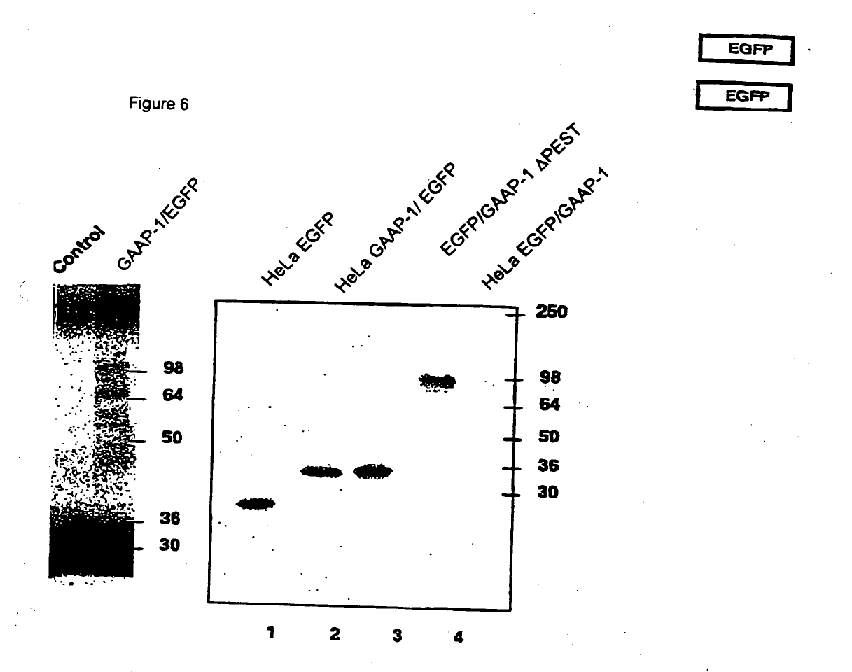

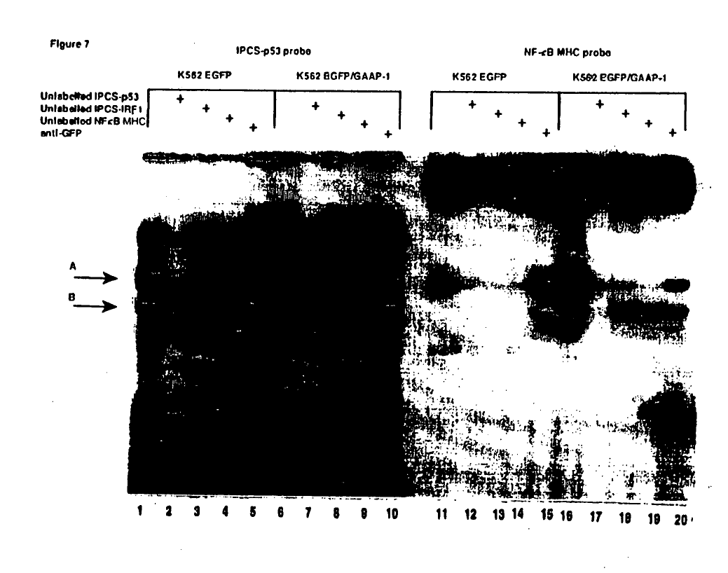

The efficiency and the specificity of GAAP-1 binding to DNA may be under the control of a post-transcriptional mechanism. In order to clarify whether GAAP-1 was subject in vivo to post-translational modifications, we performed a Western blot analysis with nuclear extracts of K562 cells stably transfected with various GAAP-1/EGFP fusion protein expression vectors (FIG. 4A) and an anti-GFP antibody. The choice of K562 cells was justified by the fact that this cell line can harbour a strong constitutive expression of GAAP-1 (see below). As shown in FIG. 6, although the in vitro translated GAAP-1/EGFP migrated at its theoretical molecular weight of 102 kD, GAAP-1/EGFP and EGFP/GAAP-1 expressed in K562 cells migrated respectively at about 35 kD and 95 kD (FIG. 6, lanes 2 and 4). As the anti-EGFP antibody detected only the N- or C-terminal portions of these fusion proteins, we can deduc that in K562 cells, GAAP-1 was cleaved at approximately 100 residues from its C-terminus. In addition, this proteolysis was independent of the PEST sequence because the GAAP-1/EGFP deleted of this sequence migrated at the same molecular weight as the wild type protein (FIG. 6, lane 3).

To analyse the effect of the intramolecular cleavege on the DNA binding properties of GAAP-1, we performed EMSAs with nuclear extracts of K562 cells stably transfected with the EGFP/GAAP-1 expression vector. Indeed, in the in vivo produced EGFP/GAAP-1 the EGFP was not released from the DNA binding part of GAAP-1, in contrast to GAAP-1/EGFP, so the fusion protein could be distinguished from endogenous GAAP-1. As shown in FIG. 7, nuclear extracts of EGFP transfected cells formed two complexes with the IPCS probe, containing probably the endogenous GAAP-1 (lane 1). Nuclear extracts of EGFP/GAAP-1 vector transfected cells gave rise to a similar pattern of migration with increased intensity mainly of band A (FIG. 7, lanes 6-9). Thus, the addition of the EGFP tag to GAAP-1 did not seem to alter the electrophoretic mobility of the DNA-protein complexes. Competition with IPCS p53 or IPCS IRF1 oligonucleotides showed that the interaction was specific (FIG. 7, lane 7 and 8), and competition with the NF-κB oligonucleotide showed that in vivo produced EGFP/GAAP-1 possessed a double specificity of binding (FIG. 7, lane 9). The formation of complex A was inhibited by an anti-GFP antibody proving that it contained EGFP/GAAP-1 (lane 10). The residual complex was probably formed by endogenous GAAP-1. Indeed, the addition of anti-EGFP antibody did not affect the formation of the complex A containing only the endogenous GAAP-1 (lane 5). The faster migrating complex B (lane 6) was also inhibited by anti-EGFP antibody, but showed a specificity lower than that of complex A (lanes 7 and 8). A control EMSA performed with the in vitro translated EGFP/GMP-1 and either the IPCS-p53 or NF-κB-MHC probe showed that the anti-EGFP antibody did not form super-shifted complexes, but rather inhibited the DNA binding activity of EGFP/GAAP-1 (data not shown). FIG. 7 presents also the results of an EMSA performed with the NF-κB probe of MHC. This experiment demonstrated that the DNA binding properties of GAAP-1 (lane 11-14) or the fusion protein EGFP/GAAP-1 (lane 16-19) were qualitatively similar on both IPCS and NF-κB probes, although an higher efficiency of binding was detected on the IPCS probe in contrast to the in vitro result.

All these data strongly suggest that in K562 cells GAAP-1 is post translationally modified by at least a proteolytic cleavage near the C-terminus.

Trancriptional Activity of GAAP-1

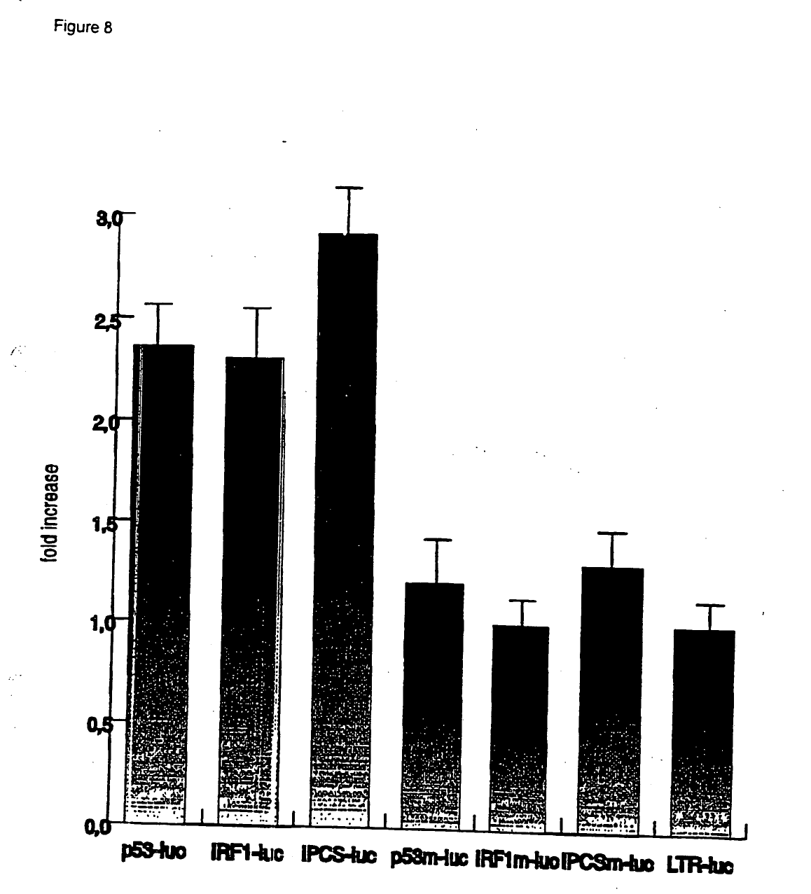

To investigate the contribution of GAAP-1 to the transcriptional activity of p53 and IRF1 promoters, we performed co-transfection analysis in K562 cells. We used plasmids containing the luciferase reporter gene under the control of p53 (p53-luc) or IRF-1 (IRF1-luc) wild type promoters or the promoters mutated at the IPCS as described previously (Lallemand 97). Alternatively, we used chimeric promoters consisting of an IPCS minimum binding site, or a mutated version of this site, cloned upstream of the SV40 promoter. Co-transfection of the GAAP-1 expression vector increased the p53-luc, IRF1-luc or IPCS-SV40-luc reporter activity approximately 2.5 times compared to a similar experiment performed using the same reporter vectors with an empty expression vector (FIG. 8). In contrast, luciferase activity expressed by constructs containing the mutated IPCS sequence was not modified by co-transfection with the GAAP-1 expression vector and was comparable to the activity obtained by co-transfection of wild type reporter vectors and an empty expression vector. These data demonstrated that GAAP-1 possessed a positive transcriptional activity. As IRF1 and p53 promoters contain several functional and putative NF-κB sites (Sims 96 Wu 94), which remained unchanged in the IPCS mutated promoters constructs, our results demonstrated that although GAAP-1 could bind the NF-κB sequence, this sequence was not implicated in the transcriptional regulation of IRF1 and p53 by GAAP-1. Lastly, we performed co-transfection experiments using an HIV-LTR/luciferase reporter construct which contains NF-κB binding sites. The failure of GAAP-1 to increase the gene reporter activity of this construct confirmed that GAAP-1 was trancriptionally active on an IPCS and not on an NF-κB sequence. This observation is in accordance with the data of Gaynor et al. which showed that the product of the 2.5 kb mRNA obtained from the differentially spliced PRDII-BF1 mRNA precursor (identical to GAAP-1) was not able to transactivate an LTR-HIV reporter construct. On the contrary, a similar experiment performed using a PRDII-BF1 expression vector resulted in an increase in LTR-HIV or NF-κB reporter construct activity (data not shown).

Involvement of GAAP-1 in Apoptosis

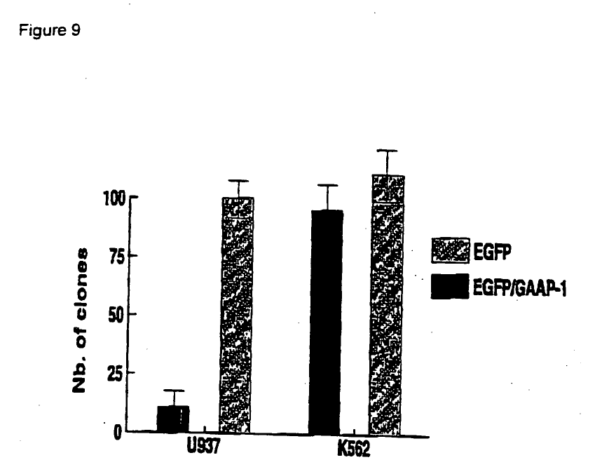

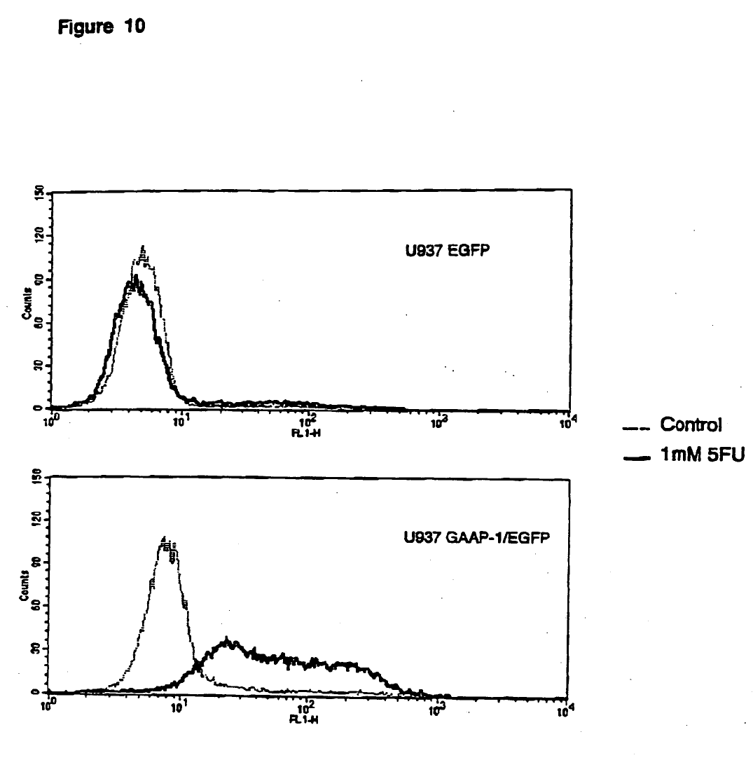

The transcriptional activation of IRF-1 and p53 promoters by GAAP-1 suggested that this factor may possess growth-suppressive and/or apoptotic properties. To test this possibility, U937 and K562 cell lines, which express both IRF-1 and p53, and neither gene respectively, were transfected with GAAP-1 or EGFP/GAAP-1 expression vectors, and tested for their colony-forming ability. The number of colonies obtained from GAAP-1 or EGFP/GAAP-1 transfected U937 cells was one tenth of that obtained from cells transfected with the EGFP control plasmid (FIG. 9). The level of fluorescence of EGFP/GAAP-1 transfected clones, determined by flow cytometry, was also much lower than that obtained with U937/EGFP cells (data not shown), suggesting that a strong over expression of GAAP-1 was lethal for these cells. In subsequent subculture steps to obtain cell lines, most of the cell clones transfected with GAAP-1 or EGFP/GAAP-1 triggered spontaneous apoptosis (FIG. 10). The clones in which the level of fluorescence was very low, seemed to proliferate in a manner similar to the parental cell line and were thus used for the following experiments.

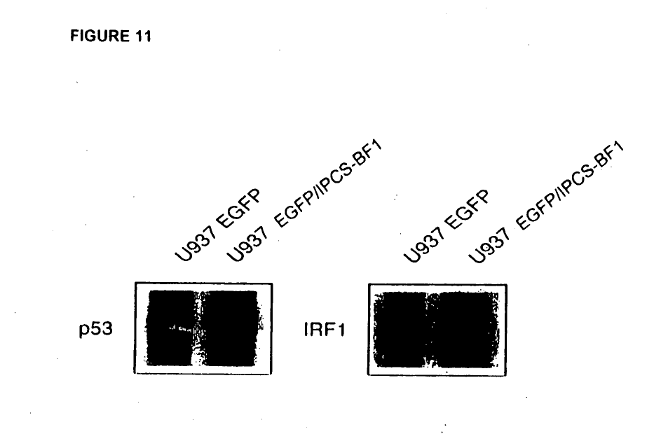

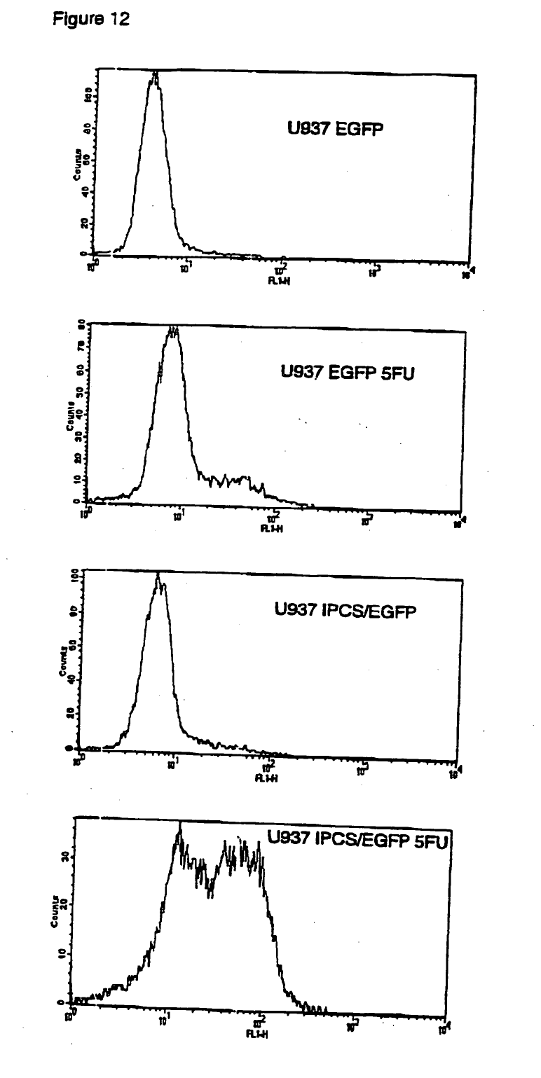

Western blot analysis of nuclear extracts showed that p53 and IRF-1 were induced 4-6 fold in the U937 EGFP/GAAP-1 cell line compared to U937 control cells (FIG. 11), demonstrating that GAAP-1 was implicated in the expression of endogenous p53 and IRF1. The increase in p53 and IRF-1 levels was correlated with a higher susceptibility of the cells to apoptosis. Treatment for 3 days with doses of the antimetabolic drug 5-fluorouracil (5FU), which were subapoptotic in U937 control cells, induced apoptosis in the U937 EGFP/GAAP-1 cell lines, as determined by TUNEL assay (FIG. 12).

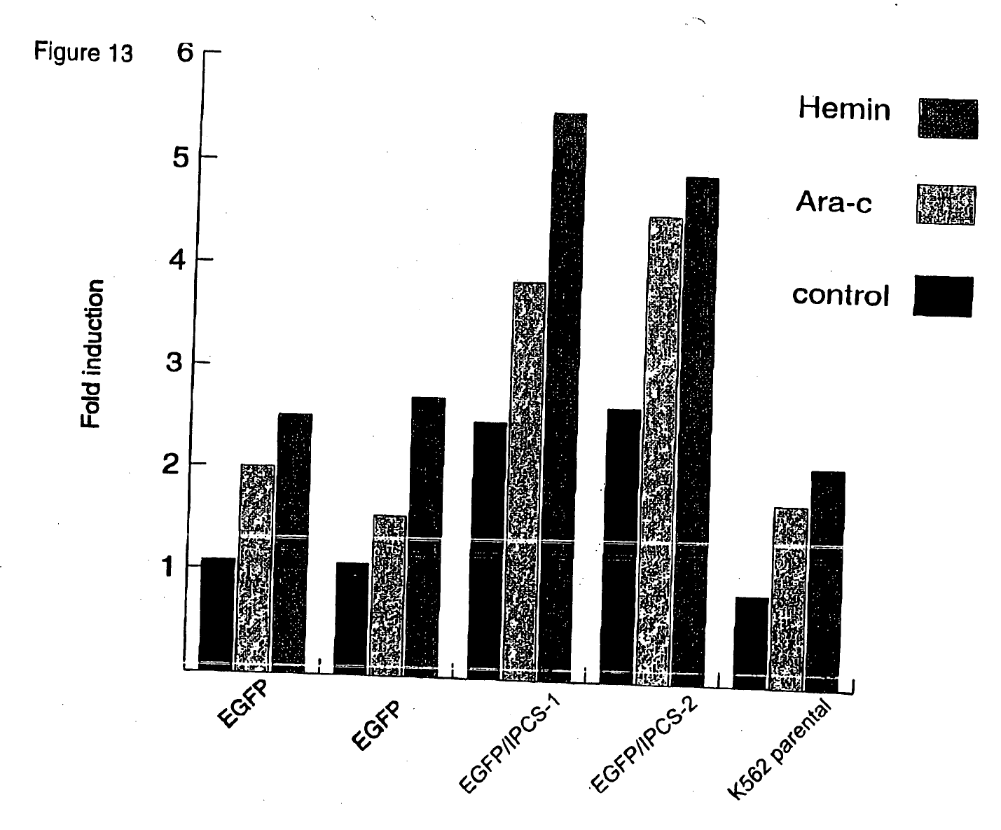

In K562 cells the number of colonies obtained with the EGFP/GAAP-1 and GAAP-1 vectors, or the control vector EGFP, was very similar. The level of expression of the fusion protein EGFP/GAAP-1, determined by flow cytometry, was heterogeneous among the clones. By Western blot analysis p53 and IRF1 were not detected in either parental or transfected cells. Cell lines expressing GAAP-1, EGFP/GAAP-1 or EGFP alone were established and their susceptibility to apoptosis was analysed. Treatment with 5FU or cytosine β-D-Arabinofuranoside (Ara-C) did not cause a significative difference in apoptosis susceptibility between GAAP-1 or EGFP/GAAP-1 transfected cells and control cells. Thus, the failure to trigger apoptosis by overexpression of GAAP-1 correlated with the lack of p53 and IRF1 genes expression in K562 cell line. However, in all K562 cell lines expressing either GAAP-1 or EGFP/GAAP-1 we observe an approximately 2-5 fold increase in the amount of hemoglobin of about 2.5 fold (FIG. 13), which was comparable to the level of hemoglobin produced by the parental clone treated with Ara-C or hemin. In addition, the expression of GAAP-1 or GAAP-1/EGFP strongly potentiated the differentiation inducing activity of Ara-C or hemin. These results indicates that enhanced expression of GAAP-1 induces erythroid differentiation and sensitises K562/GAAP-1 cells to differentiation induced by Ara-C or hemin.

Discussion

A cDNA encoding a DNA binding factor, which positively regulated the IRF-1 and the p53 promoter activity through IPCSs, was cloned by the yeast one-hybrid system. Surprisingly, this cDNA, designated IPSC-BF1 cDNA, corresponded exactly to the 2.5 kb alternative transcript of the PRDII-BF1 gene previously described by Gaynor and al (91). PRDII-BF1 was cloned independently by several groups (Fan 90, Baldwin 90, Muchardt 92) as a transcription factor implicated in the regulation of the IFNβ, MHC I promoters, and the HIV-LTR. The binding site of PRDII-BF1 on these regulatory sequences is an NF-κB recognition element. PRDII-BF1 is a zinc finger protein possessing two pairs of zinc fingers, each capable of binding individually an NF-κB site (Fan 90 Gaynor 91). We have shown that GAAP-1, which possess only one pair of fingers, is able to bind specifically to at least two kinds of unrelated DNA sequences, the IPCS and the NF-κB site. In order to elucidate this double specificity of binding, we performed EMSA using in vitro translated GAAP-1 or mutated forms of this factor with either IPCS or NF-κB probes. The destruction of either of the two zinc fingers eliminates the binding to IPCS or NF-κB sequences, demonstrating that both zinc finger structures are required for both binding activities. This result suggests that amino acids others than those engaged in zinc finger formation (His and Cys residues) are implicated in the specific binding with the NF-κB or the IPCS motifs. A multiple specificity of DNA binding has already been reported for the WT1 proteins, whose multiple specificity relies on amino acid residues modified by alternative splicing between two zinc fingers (Hewitt 96). To our knowledge, GAAP-1 represents the first description of a zinc finger protein having fingers with a double specificity of binding.

The in vitro translated GAAP-1 possesses an affinity for the IPCS probe lower than that for the NF-κB probe. However, in vivo produced GAAP-1 tagged with EGFP has the opposite property, suggesting that GAAP-1 is subjected to post translational modification which increases specifically its affinity for IPCS. Western blot analysis performed on the in vivo produced fusion proteins GAAP-1/EGFP-BF1 and EGFP/GAAP-1 suggests that at least one mechanism may be implicated in the control of GAAP-1 binding affinity. Indeed, this experiment clearly demonstrates that a proteolytic cleavage occurs in vivo at approximately 100 residues from the C terminus of GAAP-1 which could be determinant for the binding properties of the protein. Thus, our data may explain the results obtained by Gaynor et al., who showed that the in vitro product of the differentially spliced 2.5 kb mRNA, which corresponds to GAAP-1, has a strong binding activity for a NF-κB sequence. These observations suggest that the physiological role of GAAP-1 requires post-translational modifications.

The activity of GAAP-1 is also regulated by its sub-cellular localisation. Although previous studies (Fan 90) have localised PRDII-BF1 exclusively in the nucleus, we have demonstrated that GAAP-1 resides in both the nucleus and cytoplasm. This difference may be ascribed to the alternative use of the two NLS present in the PRDII-BF1 gene. The first NLS was identified by Fan in PRDII-BF1 and is not encompassed by GAAP-1. This NLS may be responsible for the strictly nuclear localisation of PRDII-BF1. We have identified a second NLS, without which GAAP-1 cannot be imported in the nucleus. This observation underlines the fact that GAAP-1 is a transcription factor possessing its own NLS and functionally independent of PRDII-BF1. While the whole GAAP-1 is localised both in the nucleus and cytoplasm, the protein deleted of the PEST domain is strictly nuclear. The PEST sequence is recognised by ubiquitin-conjugating enzymes in the pathway which leads proteins to be degraded in the proteasome (Rechsteiner 96). Furthermore, the PEST domain may also be required for the export of transcription factors from the nucleus to the cytoplasm in order to be degraded in the proteasome (ref Jab1). Thus, the deletion of the PEST domain may retain GAAP-1 into the nucleus, by abolishing the export of the protein.

Although PRDII-BF1 has been identified on the basis of its interaction with the IFNβ promoter (Fan 90) and the enhancers of MHC I and kappa Immunoglobulin genes (Baldwin 90), its transcriptional activity was demonstrated only in the case of the HIV enhancer (Tsujimoto 91,). In addition, it was demonstrated that the 90 kD and the 70 kD proteins, the last one corresponding to GAAP-1, which are coded by two differentially spliced messages of the PRDII-BF1 gene, are devoid of any transcriptional activity on the HIV LTR (Gaynor). This observation is in agreement with our data and suggests that GAAP-1, contrary to PRDII-BF1, is not engaged in physiological transcription involving NF-κB binding sites. Our results, indeed, unambiguously demonstrate a positive transcriptional activity of GAAP-1 on the p53 and IRF1 promoters, which is strictly correlated to the presence of an IPCS binding site on these promoters. Furthermore, this transcriptional activity is confirmed by the enhancement of the p53 and IRF1 endogenous genes products shown in the U937 cells transfected with a GAAP-1 expression vector. Thus, all these data permit us to define GAAP-1 as a transcription factor implicated in the positive transcription of at least the two anti-oncogenes IRF1 and p53. This property suggests that GAAP-1 may also act as an anti-oncogene.

U937 cells over expressing GAAP-1 undergo apoptosis with a susceptibility which correlates with the levels of p53 and IRF1. This result suggests that the possible cause of cellular death is the increase of p53 and IRF1. It has to be pointed out that in U937 cells the p53 gene contains point mutations (ref). However, a transitory increase in the expression of the endogenous p53 was implicated (Gabriele 99) or is even required (Yeung 98) for the apoptosis of these cells, suggesting that p53, in spite of its mutations, is still active. Moreover, the increase in IRF1 expression may also contribute to apoptotic death, as has been already reported (Nguyen 97). On the other hand, U937 cell lines which express levels of GAAP-1 compatible with cell proliferation Exhibit increased sensitivity to genotoxic drugs. Thus, GAAP-1 may play an important role in the surveillance of the genomic integrity controlling the level of the key component p53.

K562 cells, which do not express either IRF1 or p53, do not trigger apoptosis following GAAP-1 over-expression, but und rgo rythroid diff rentiation. This result suggests that GAAP-1 also regulates the expression of unknown genes implicated in other cellular functions.

In conclusion, our data suggest that the targets of the transcription factor GAAP-1 are multiple and are involved in processes concerning cell growth control, apoptosis and differentiation.

Isolation and Characterization of GAAP-2

The transcript corresponding to GAAP-2 was observed by multiplex-RT-PCR carried out on mRNA obtained from various human organs such as testis, bone marrow, lungs, colon, placenta and PBLs.

The obtained DNA has been cotransfected in U937 cells and in K562 cells, leading to transactivating properties of GAAP-2 on the promoters of p53 and IRF1, similar to those observed with GAAP-1.

The intracellular localization of GAAP-2 has been studied with a fusion protein EGFP/GAAP-2 and is similar to the localization of GAAP-1, i.e., nuclear and cytoplasmic.

References

Nguyen, H., Lin, R., and Hiscoft, J. (1997). Activation of multiple growth regulatory genes following inducible expression of IRF1 or IRF1/ReIA fusion protein. Oncogen 15, 1425-1435.
Feintein, E., Gale R. P., Reed, J., and Canaani, E. (1992). Expression of the normal p53 genes induces differentiation of k562 cells. Oncogene 7, 1853-1857
Chylicki, K., Ehinger, M., Svedberg, H., Bergh, G., Olsson, I., and Gullberg, U. (2000). P53-mediated differentiation of the erythroleukemia cell line K562. Cell growth and differentiation. 11, 315-324
Ray, S., Bullock, G., Nunez, G., Tang, C., Ibrado, A. N., Huang, Y., and Bhalla, K. (1996). Enforced expression of BcI-Xs induces differentiation and sensitizes chronic myelogenous leukemia-blast crisis K562 cells to 1-b-d-arabinofuranosylcytosine mediated differentiation and apoptosis. Cell growth & differentiation 7, 1617-1623
Fan, C. M., and Maniatis, T. (1990). A DNA-binding protein containing two widely separated zinc finger motifs that recognize the same DNA sequence. Genes and Development 4, 29-42.
Hewitt, S. M., Fraizer, G. C., Wu, Y. J., Rauscher III, F. J., and Saunders, G. F. (1996). Differential function of Wilms' tumor gene WT1 splice isoforms in transcriptional regulation. J. Biol. Chem. 271, 8588-8592.
Hewitt S M, Saunders G F. Differentially spliced exon 5 of the Wilms' tumor gene WT1 modifies gene function. Anticancer Res. 1996 16(2): 621-6.
Raman, V., Martensen, S. A., Reisman, D., Evron, E., Odenwald, W. F., Jaffee, E., Marks, J., and Sukumar, S. (2000). Compromised HOXA5 function can limit p53 expression in human breast tumours. Nature 405, 974-978.
Lallemand, C., Bayat-Sarmadi, M., Blanchard, B., and Tovey, M. G.(1997). Identification of a NovelTranscriptional RegulatoryElement Common to the p53 andInterferon Regulatory Factor1 Genes. J. Biol. Chem. 272, 29801-29809.
Baldwin jr., A. S., LeClair, K. P., Singh, H., and Sharp P. A. (1990). A Large protein containing zinc finger domains binds to related sequence element in the enhancer of the class I major histocompatibility complesx and kappa immunoglobulin genes. Mol. Cell. Biol. 10, 1406-1414.
Michael C. Yeung and Allan S. Lau Tumor Suppressor p53 as a Component of the Tumor Necrosis Factor-induced, Protein Kinase PKR-mediated Apoptotic Pathway in Human Promonocytic U937 Cells J Biol Chem, Vol. 273, Issue 39, 25198-25202, Sep. 25, 1998
Gabriele, L., Phung, J., Fukumoto, J., Segal., D., Wang, I. M., Giannakakou,. P., Giese, N. A., Ozato, K., and Morse III, H. C. (1999). Regulation of apoptosis in myeloide cells by interferon concensus sequence-binding protein. J. Exp. Med. 190, 411-421.
Fisher, R. C., Olson, M. C., Pongubala, J. M. R., Perkel, J. M., Atchison, M. L., Scott, E. W., and Simon, M. C. (1998). Normal myeloid development requeres both the glutamine rich transactivation domain and the PEST region of transcription factor PU.1 but not the potent acidic transactivation domain. Mol. Cell. Biol, 18, 4347-4357.
Pongubabla, J. M., Van Bevere,n C., Nagulapalli, S., Klemsz, M., McKercher, S., Maki, R., and Atchison, M. L. (1993). Effect of PU.1 phosphorylation on interaction with NF-EM5 and transcriptional activation. Science 259, 1622-1625
Shaw, P., Bovey, R., Tardy, S., Sahli, R., Sordat, B., and Costa, J. (1992) Proc. Nati. Acad. Sci. U.S.A. 89, 4495-4499
Yonish-Rouach, E., Resnitzky, D., Lotem, J., Sachs, L., Kimchi, A., and Oren, M. (1991) Nature 352, 345-347 Wt-p53 action in human leukaemia cell lines corresponding to different stages of differentiation.
Rizzo M G, Zepparoni A, Cristofanelli B, Scardigli R, Crescenzi M, Blandino G, Giuliacci S, Ferrari S, Soddu S, and Sacchi, A. Br J Cancer May 1998 77:9 1429-38
Soussi T The p53 tumor suppressor gene: from molecular biology to clinical investigation. Ann N Y Acad Sci June 2000;910:121-37; discussion 137-9
Xu G, Sze S H, Liu C P, Pevzner P A, Amheim N Gene hunting without sequencing genomic clones: finding exon boundaries in cDNAs. Genomics Jan. 15, 2000;47(2): 171-9
Seeler J S, Muchardt C, Suessle A, Gaynor R B. Transcription factor PRDII-BF1 activates human immunodeficiency virus type 1 gene expression. J Virol. February 1994; 68(2): 1002-9.
Muchardt C, Seeler J S, Ninula A, Shurland D L, Gaynor R B. Regulation of human immunodeficiency virus enhancer function by PRDII-BF1 and c-rel gene products. J Virol. January 1992;66(1):244-50.
Vogelstein B., Lane D., Levine A. J. Surfing the p53 network. 2000 Nature 408, 307-310
iwase S., Furukawa Y, Kikuchi J, Saito S, Nakamura M, Nakayama R, Horiguchi-Yamada J, Yamada H. Defective binding of IRFs to the intiator element of interleukin-1beta-converting enzyme (ICE) promoter in an interferon-resistant Daudi subline. FEBS Lett. May 7, 1999, 450(3): 263-7.
Bressac B, Galvin K M, Liang T J, Isselbacher K J, Wands J R, Ozturk M. Abnormal structure and expression of p53 gene in human hepatocellular carcinoma. Proc. Natl. Acad. Sci. USA. March 1990; 87(5): 1973-7.
Rechsteiner M., and Rogers S. W. PEST sequences and regulation by proteolysis. TIBS. 21, 267-271 (1996).
Maziere C., Meignotte A, Dantin F. Conte M A, Maziere J C. Oxidized LDL induces an oxidative stress and activates the tumor suppressor p53 in MRC5 human fibroblasts. Biochem Biophys Res Commun. Sep. 24, 2000;276(2): 718-23.
Gaynor, R. B., Muchardt, C., Diep, A., Mohandas, T. K., Sparkes, R. S., & Lusis, A. J. Genomics 9, 758-761 (1991).
Sims, S. H., Cha, Y., Romine, M. F., Gao, P. Q., Gottlieb, K., Deisseroth, A. B. Mol. Cell Biol. 13, 690-702 (1993).
Wu, H. & Lozano, G. J. Biol. Chem. 269, 20067-20074 (1994).

1-45. (canceled)

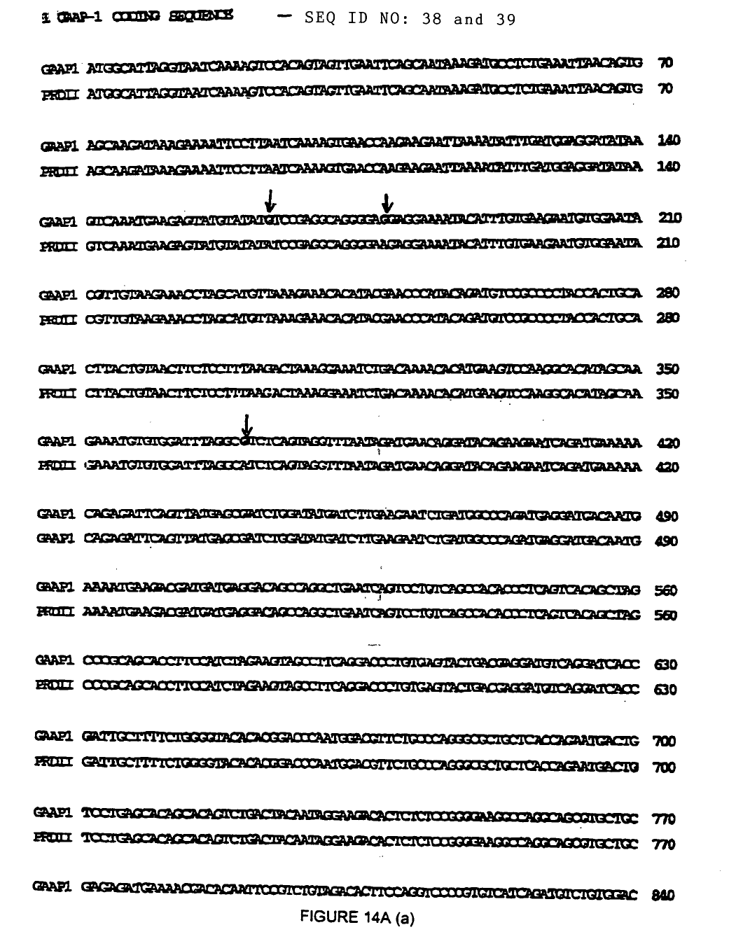

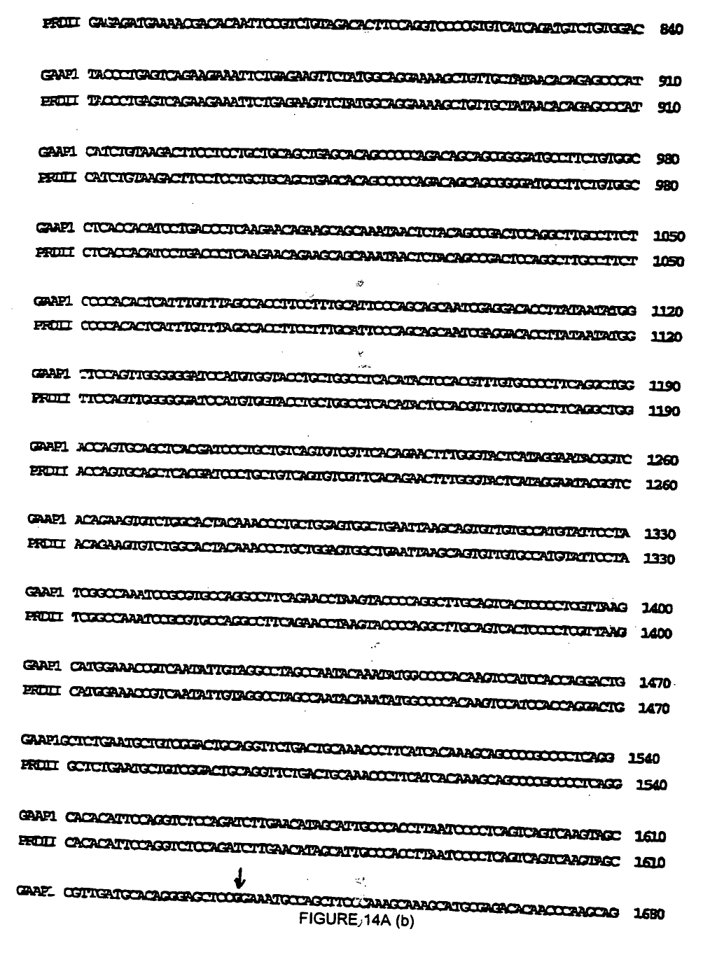

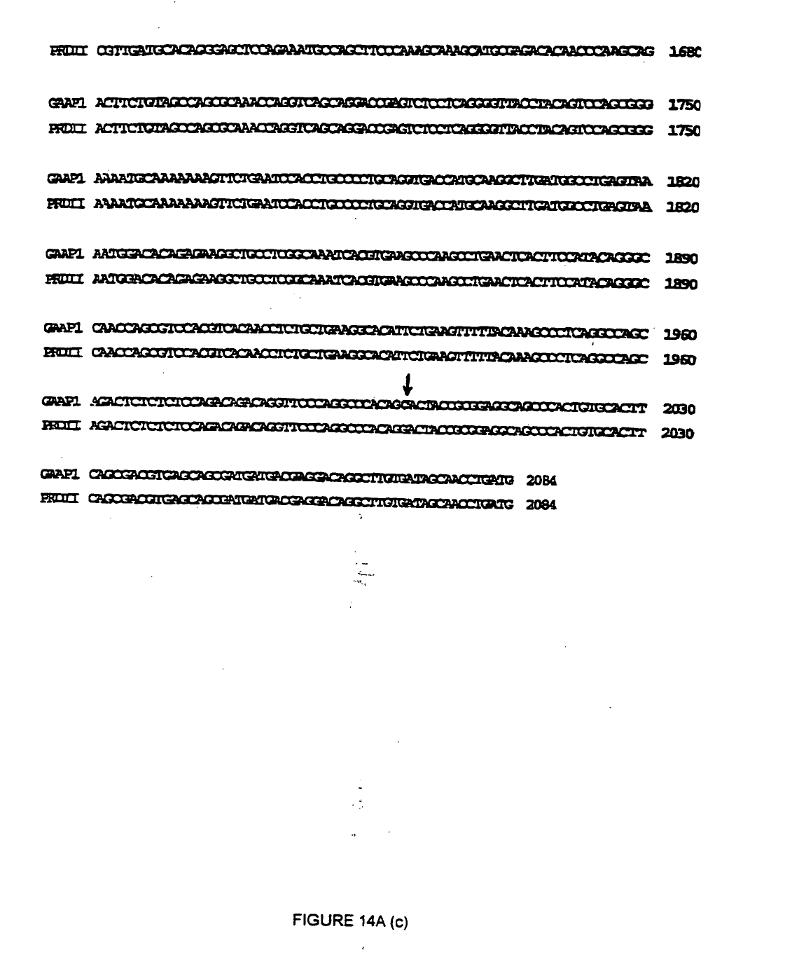

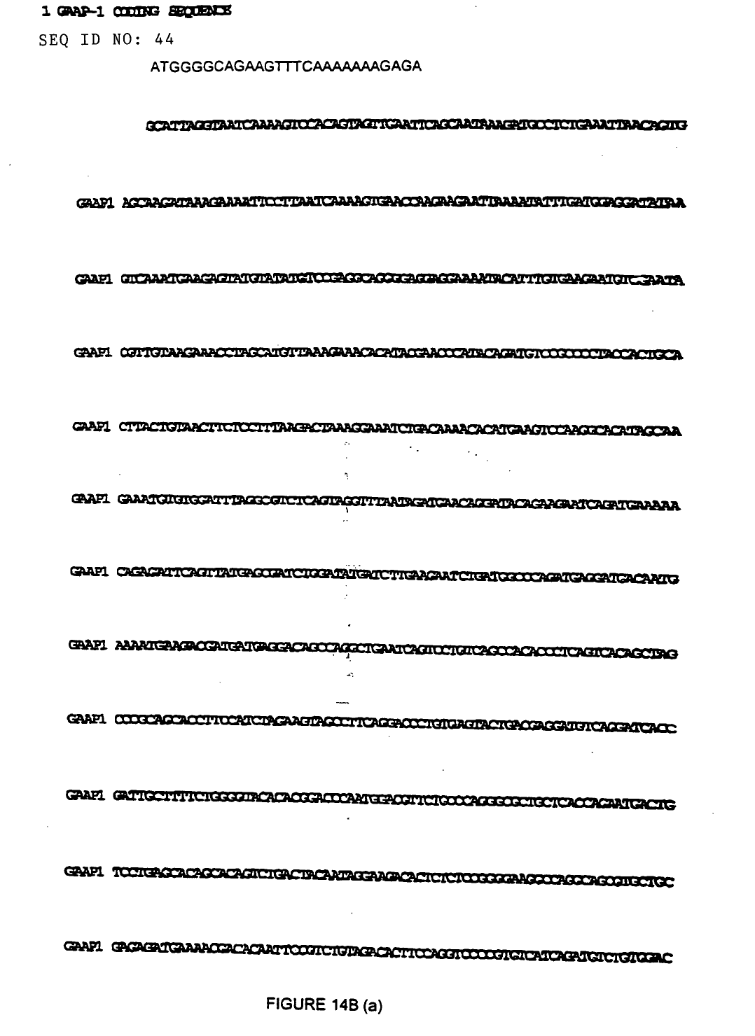

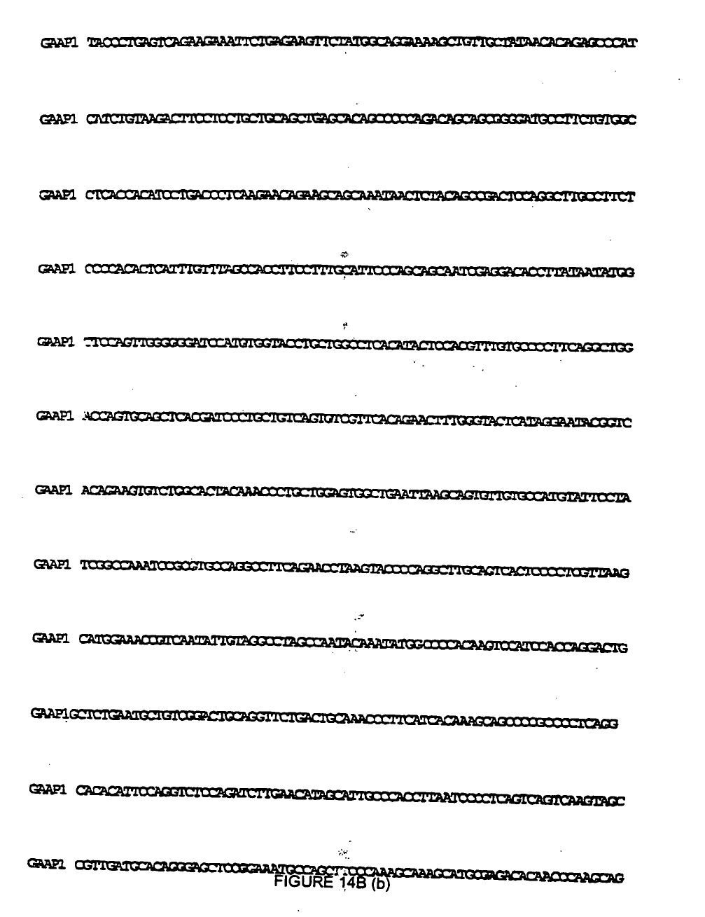

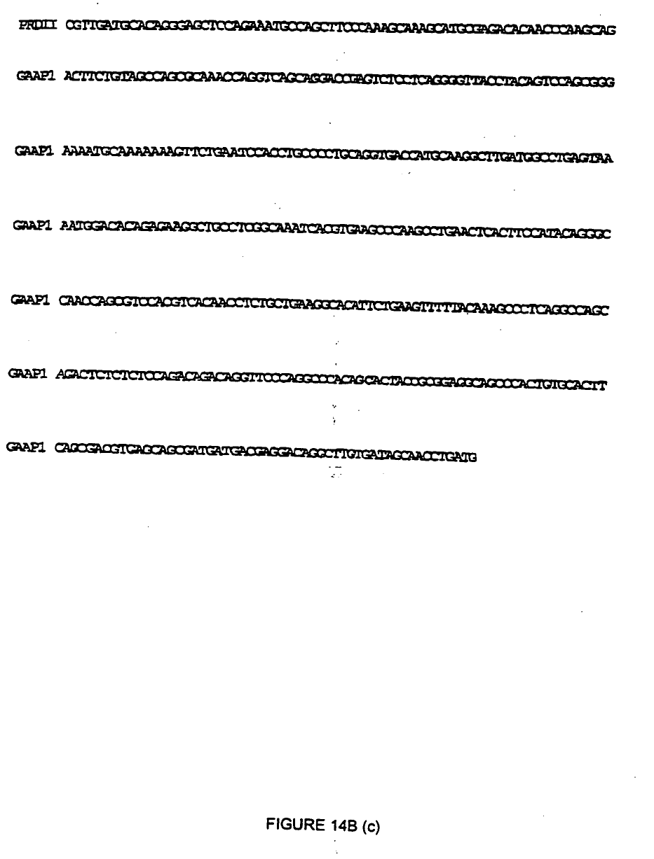

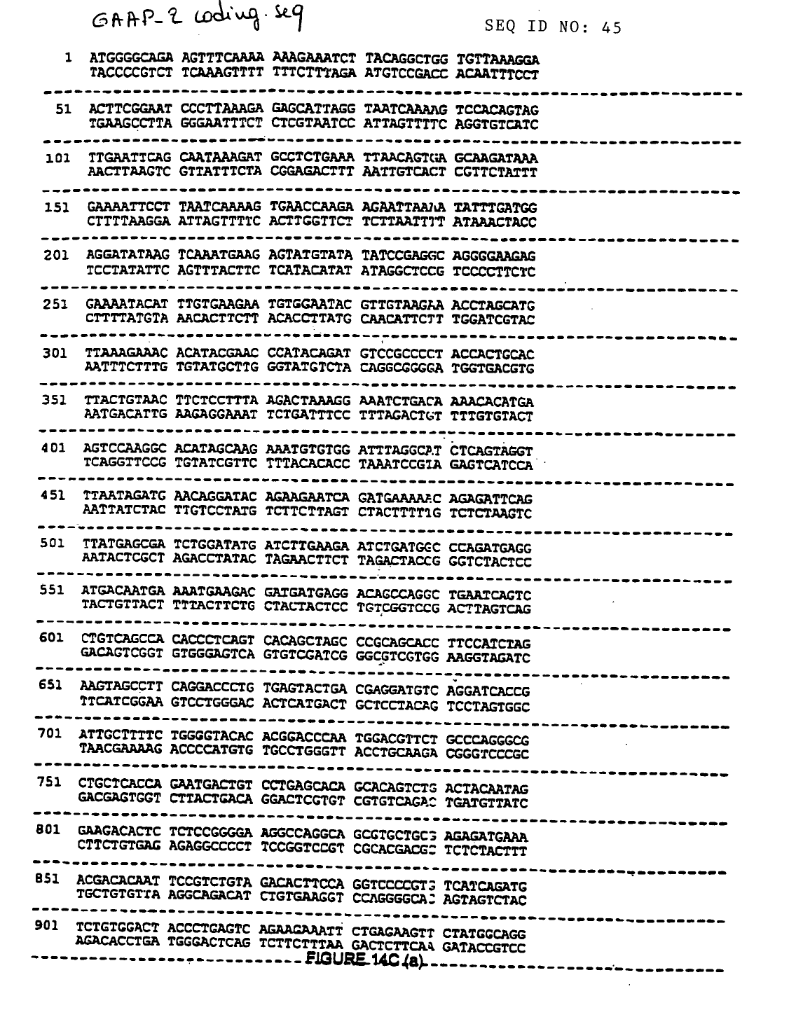

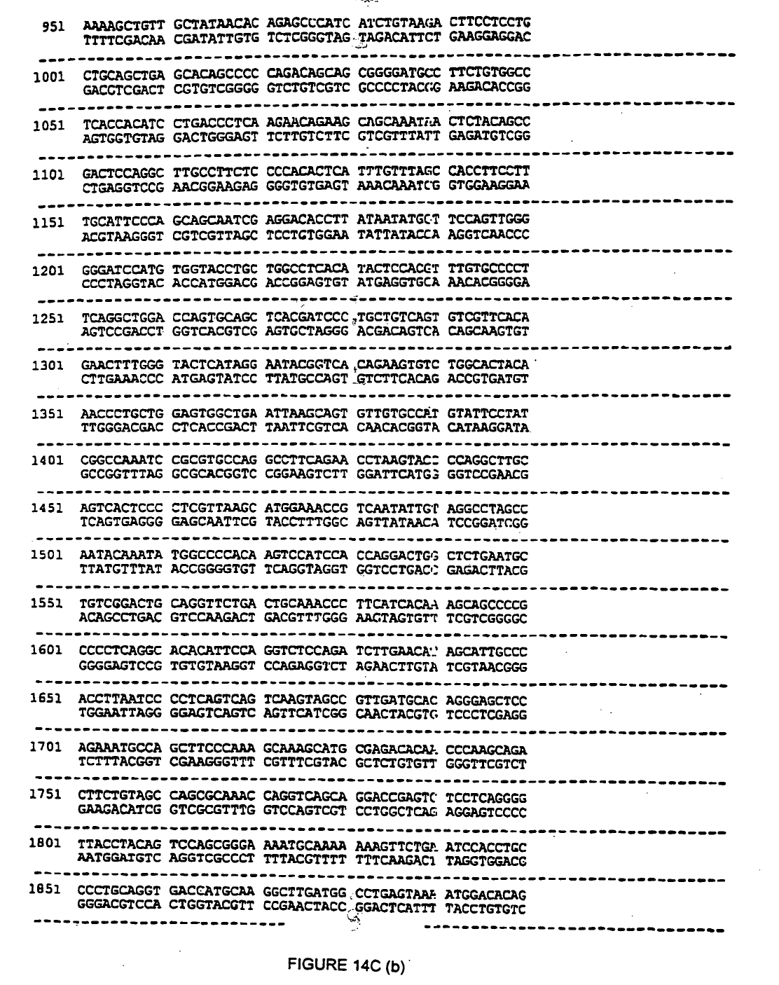

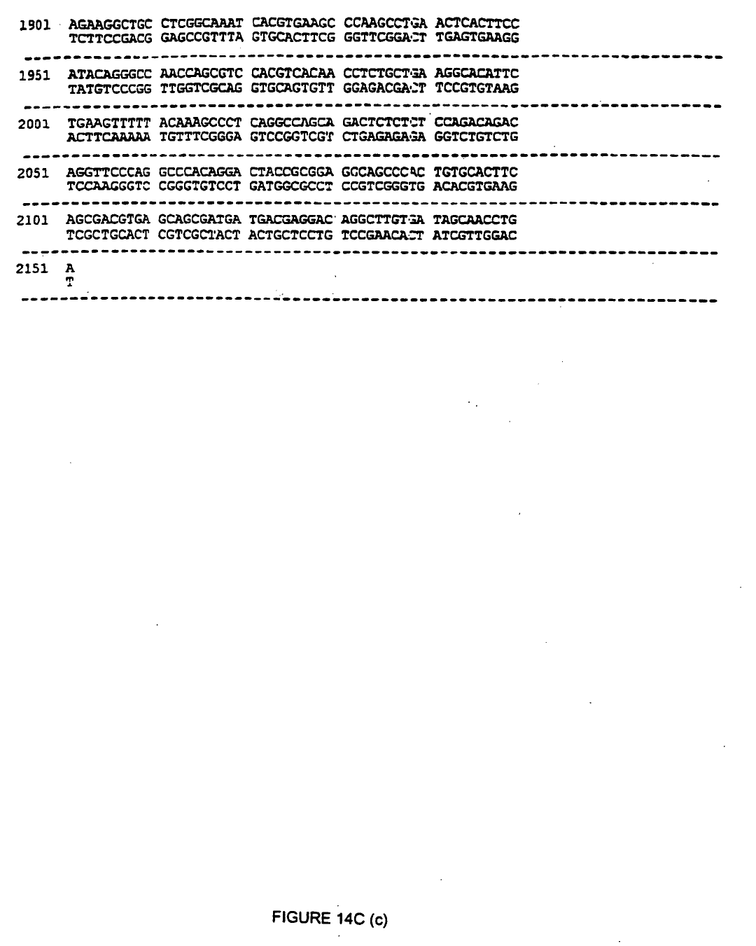

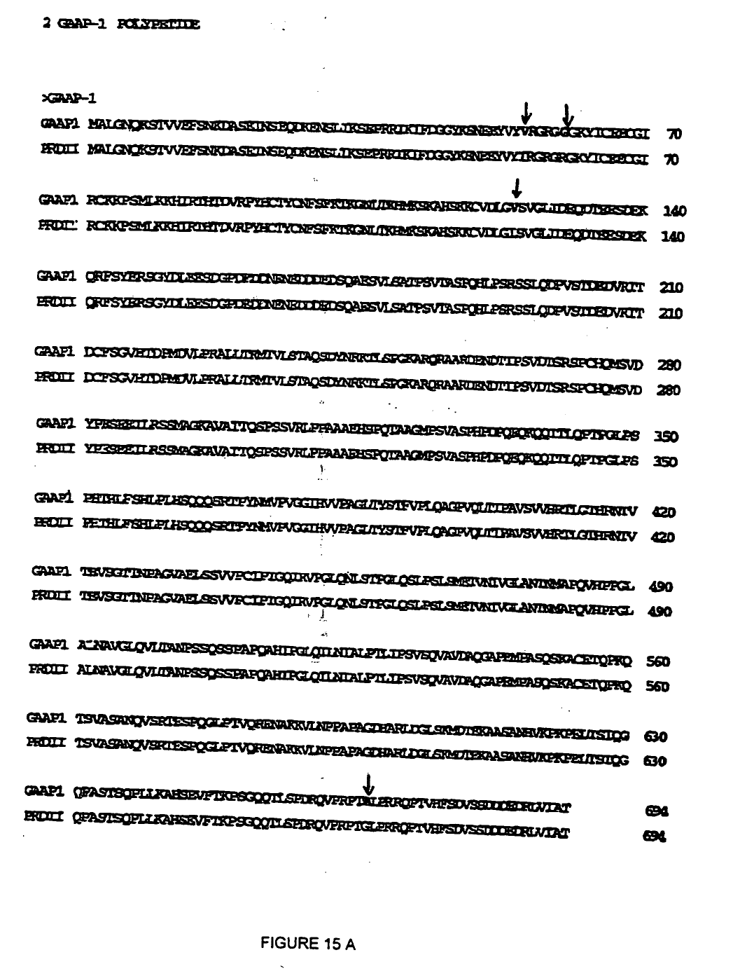

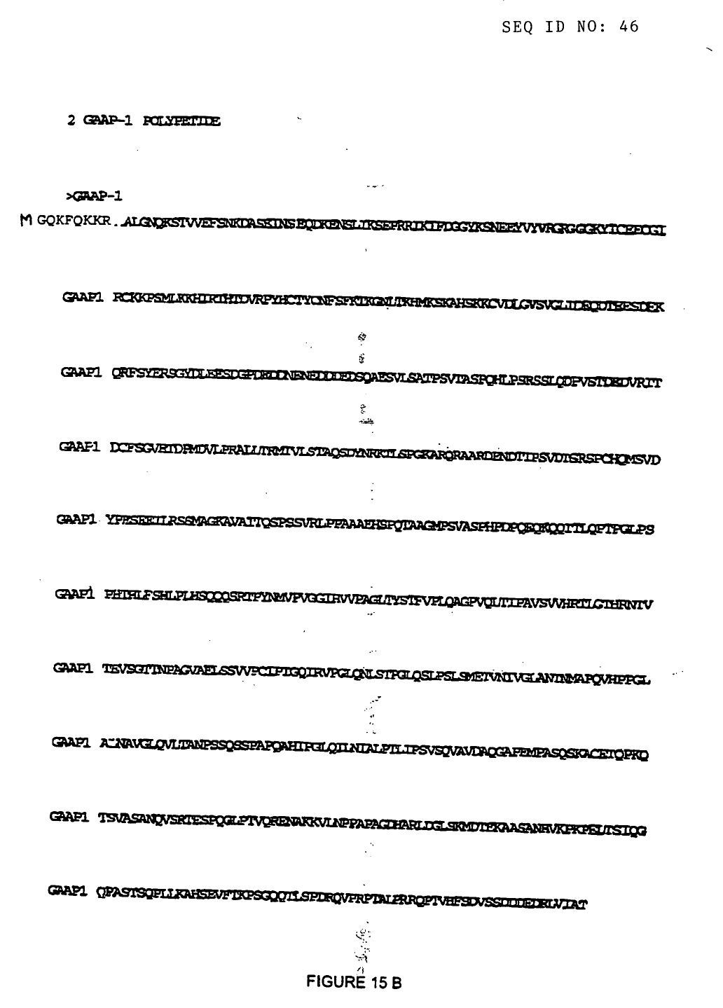

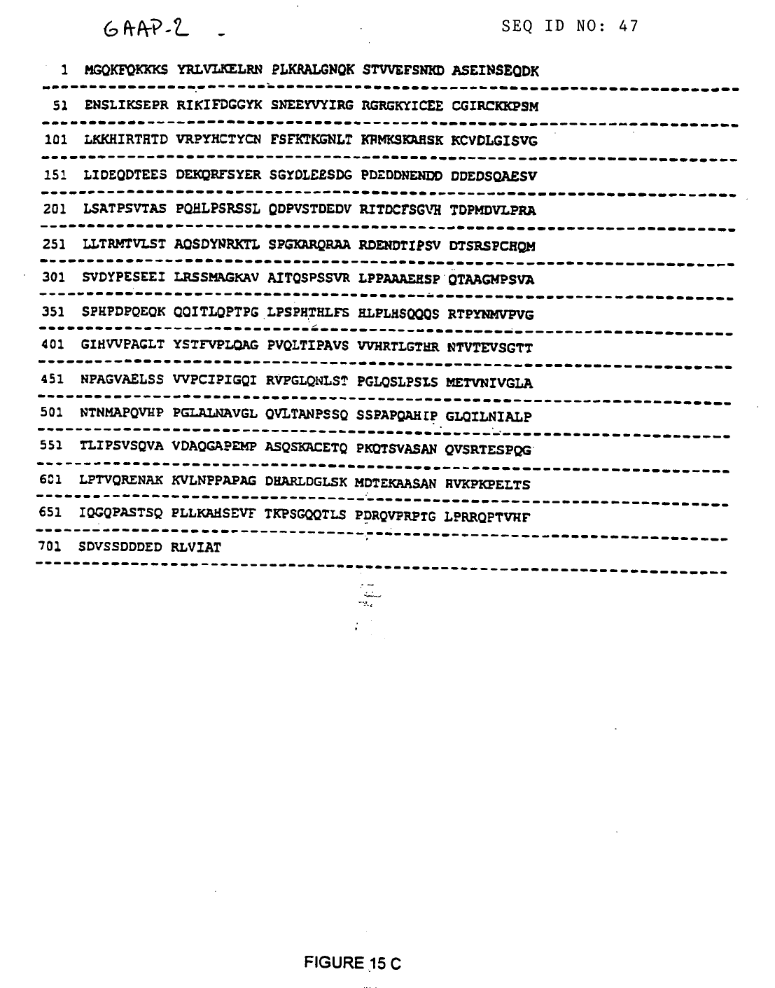

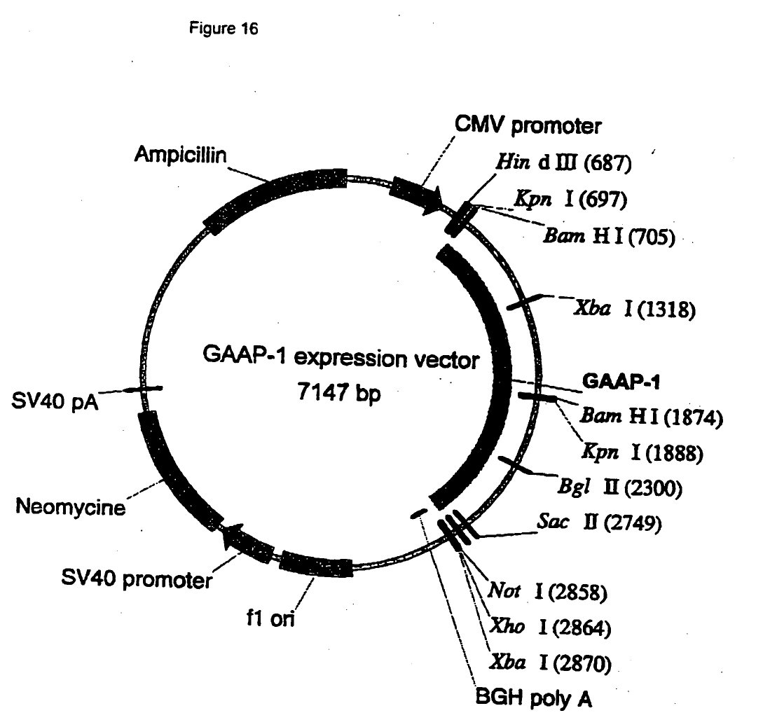

46. A method for expressing in a determined eucaryotic cell, a polypeptide capable of interacting with a nucleotide sequence designated IPCS which comprises the DNA sequence AAATGNNNNC (SEQ ID NO: 2), wherein N means any nucleotide (G, A, C or T(U)), and capable of acting as a positive transcription factor for the transcription of a nucleotide sequence placed under the control of said (PCS sequence and present in said eucaryotic cell, comprising expressing in said eukaryotic cell a polynucleotide comprising a nucleotide sequence selected from the group consisting of:
(i) a nucleotide sequence comprising the DNA sequence of SEQ ID NO: 38, and represented in FIG. 14A, or of SEQ ID NO: 44 and represented in FIG. 14B;
(ii) a nucleotide sequence encoding a polypeptide having the amino-acid sequence of SEQ ID NO: 40, and represented in FIG. 15A or encoding a polypeptide having the amino-acid sequence of SEQ ID NO: 46 and represented in FIG. 15B;
(iii) a nucleotide sequence comprising the DNA sequence of SEQ ID NO: 45, and represented in FIG. 14C;
(iv) a nucleotide sequence encoding a polypeptide having the amino-acid sequence of SEQ ID NO: 47, and represented in FIG. 15C;
(v) a nucleotide sequence derived from sequence defined under (i), (ii), (iii) or (iv) wherein said sequence is modified particularly by deletion, addition or substitution of one or more nucleotides providing that the resulting nucleotide sequence encodes a polypeptide capable of binding said nucleotide sequence designated IPCS.

47. The method of claim 46 wherein the expressed polypeptide acts as a positive transcription factor for a gene involved in a process selected from the group consisting of control of cellular growth, cellular proliferation, cellular differentiation or cellular apoptosis.

48. The method according to claim 46, wherein the nucleotide sequence comprises the cDNA corresponding to a 2.5 kb coding sequence of the transcript of the PRDII-BF1 gene.

49. The method according to claim 48, wherein a start codon is added upstream from exon III of the PRDII-BF1 gene.

50. The method according to claim 48, wherein the nucleotide sequence comprises the succession of exons III, V, VI, VII, VIII and IX of the PRDII-BF1 gene, said nucleotide sequence being devoid of exon IV of said PRDII-BF1 gene in the case of GAAP-1 and containing the first 45 nucleotides of exon IV in the case of GAAP2.

51. The method according to claim 46 in which the nucleotide sequence codes for a GAAP-1 polypeptide comprising the amino acid sequence of SEQ ID NO: 40 or 46 or for a GAAP-2 polypeptide comprising the amino acid sequence of SEQ ID NO: 47.

52. the method according to claim 46, wherein the nucleotide sequence codes for a variant of the GAAP-1 polypeptide or the GAAP-2 polypeptide, said variant being derived from GAAP-1 or GAAP-2 by insertion, deletion or substitution of one or several amino acid residues, provided it retains the property of GAAP-1 or GAAP-2 to bind an IPCS sequence and to act as a transcriptional factor for the for the transcription of a nucleotide sequence placed under the control of said IPCS sequence and present in a eucaryotic cell.

53. The method according to claim 46, in which the nucleotide sequence hybridizes under stringent conditions with the DNA sequence of SEQ ID NO: 38 or 44 or with the DNA sequence of SEQ ID NO: 45.

54. The method according to claim 46, wherein said nucleotide sequence is placed under the control of a promoter sequence selected among constitutive or inducible promoters.

55. The method according to claim 46, wherein the eucaryotic cells are malignant cells.

56. The method according to claim 46, wherein the eucaryotic cells are those of a developed tumor.

57. The method according to claim 46, for the control of cell apoptosis.

58. The method according to claim 46, wherein the positive regulation of transcriptional activity is obtained for a gene
selected from the group consisting of p53, IRF1, Rb, p21 (WAF1), p27, wt1, bax, TNF receptor and FAS.

59. The method according to claim 46, wherein the positive regulation of transcriptional activity allows regulation of transcription of several genes in said eukaryotic cell.

60. A polynucleotide comprising the nucleotide sequence comprising the DNA sequence of SEQ ID NO: 38, represented in FIG. 14A, said nucleotide-sequence being devoid of the sequence forming exon IV in the PRDII-BF1 gene.

61. The polynucleotide of claim 60 which consists of the DNA sequence of SEQ ID NO: 38.

62. The polynucleotide according to claim 60 which is selected from the group consisting of:
(i) a fragment of the DNA sequence of SEQ ID NO: 38 (FIG. 14A), or a fragment of the DNA sequence of SEQ ID NO: 44 (FIG. 14B) which can be used as a specific probe to detect the presence of said DNA sequence of SEQ ID NO: 38, 44 or 45 or a mutated sequence thereof, in a sample,
(ii) a nucleotide sequence encoding a polypeptide having the amino-acid sequence of SEQ ID NO: 40, or a nucleotide sequence encoding a polypeptide having the amino-acid sequence of SEQ ID NO: 46 or a nucleotide sequence encoding a polypeptide having the amino acid sequence of SEQ ID NO: 47,
(iii) a nucleotide sequence derived from sequence defined under (i) or (ii) wherein said sequence is modified, especially by deletion, addition or substitution of one or more nucleotides providing that the resulting nucleotide sequence encodes a polypeptide capable of binding a nucleotide sequence designated IPCS which comprises the DNA sequence AAATGRYKKC (SEQ ID NO: 15), and is capable when used in appropriate conditions, of expressing in a determined eucaryotic cell, a polypeptide interacting with the nucleotide sequence designated IPCS and acting as a positive transcriptional factor for the transcription of a nucleotide sequence placed under the control of said IPCS sequence and present in said eucaryotic cell.

63. A polynucleotide comprising the nucleotide sequence which is contained on plasmid pGAAP1 deposited at the ECACC under accession no. 01052921.

64. A recombinant polypeptide that is the product of the expression in a eucaryotic cell of a nucleotide sequence coding for a polypeptide capable of interacting with the nucleotide sequence designated IPCS and capable of acting as a positive transcription factor for the transcription of a nucleotide sequence placed under the control of said IPCS sequence and present in said eucaryotic cell.

65. A recombinant polypeptide, being the product of the expression in a eucaryotic cell, of a nucleotide sequence according to claim 60.

66. A recombinant polypeptide according to claim 64 which regulates the transcriptional activity of a gene selected among p53 and IRF1 when it is expressed in a eucaryotic cell constitutively expressing said gene.

67. A recombinant polypeptide according to claim 65 which regulates the transcriptional activity of a gene selected among p53 and IRF1 when it is expressed in a eucaryotic cell constitutively expressing said gene.

68. A recombinant polypeptide according to claim 64 which has an apparent molecular weight of 75 kDa by SDS PAGE electrophoresis.

69. A recombinant polypeptide, being the expression product in a mammalian cell, of a nucleotide sequence according to claim 60, said recombinant polypeptide including post translational modification enabled in said eucaryotic cell.

70. The recombinant polypeptide according to claim 64 which comprises the amino acid sequence of SEQ ID NO: 40, 46, or 47.

71. The recombinant polypeptide according to claim 64 which comprises a fragment of the amino acid sequence of SEQ ID NO: 40 provided the polypeptide is capable of interacting with the nucleotide sequence designated IPCS to act as a positive transcription factor for the transcription of a nucleotide sequence placed under the control of said IPCS sequence and present in said eucaryotic cell.

72. A recombinant eucaryotic cell comprising an inserted polynucleotide comprising the nucleotide sequence according to claim 60.

73. The recombinant eucaryotic cell according to claim 72 which expresses the polypeptide encoded by the inserted nucleotide sequence.

74. The recombinant eucaryotic cell according to claim 72 which is a cell expressing genes selected from the group consisting of genes involved in the control of cell growth, genes involved in cell differentiation, genes involved in cell proliferation and genes involved in cell apoptosis.

75. The recombinant eucaryotic cell according to claim 72 which is a malignant cell.

76. A recombinant eucaryotic cell comprising an inserted polynucleotide encoding a polypeptide capable of binding a nucleotide sequence designated IPCS and comprising the DNA sequence AAATGNNNNC (SEQ ID NO: 2), to enable, in appropriate conditions, the expression of a polypeptide capable of interacting with the nucleotide sequence designated IPCS and capable of acting as a positive transcriptional factor for the transcription of a nucleotide sequence placed under the control of said IPCS sequence and present in said eucaryotic cell.

77. The recombinant eucaryotic cell according to claim 72, wherein the polypeptide comprises the amino acid sequence of SEQ ID NO: 40 or 46 or 47.

78. The recombinant eucaryotic cell according claim 72, which is selected from the group consisting of U937, K562, SK—N—,SH, MCF7, KG1 and TF1.

79. A recombinant vector comprising a polynucleotide according to claim 60.

80. The recombinant vector according to claim 79 which is an expression vector suitable for expression of said polynucleotide in a eucaryotic cell.

81. The recombinant vector according to claim 80 which is suitable for transient or controlled expression of said polynucleotide.

82. The recombinant vector according to claim 80, wherein said polynucleotide is placed under the control of a promoter regulated by a physiologically acceptable compound.

83. The recombinant vector according to claim 80, wherein the transcription of said insert is placed under the control of an exogenous transactivating system.

84. The recombinant vector according to claim 80, which is suitable for gene therapy.

85. The recombinant vector according to claim 84, which is selected from the group consisting of viral, retroviral, lentiviral, poxviral, adenoviral, AAV vectors.

86. A composition suitable for a therapeutic use which comprises a polynucleotide according to claim 60 or a recombinant cell according to claim 72, or a recombinant polypeptide according to claim 64.

87. A method of treating a subject comprising administering to said subject the composition according to claim 86 in combination with an antiviral agent or an anticancer agent.

88. The method according to claim 87 wherein the anticancer agent is an immuotherapeutic agent, a chemotherapeutic agent or is radiotherapy.

89. The method according to claim 87, for the treatment of a malignant cell.

90. A method for the in vitro detection of a deficient BRDII-BFI gene comprising the steps of:
contacting a probe comprising a polynucleotide having the nucleotide sequence of SEQ ID NO: 38, 44, or 45, or a fragment thereof comprising the zinc finger binding domains corresponding to the domains localized within exon VI of the BRDII-gene, with the DNA of a cell normally constitutively expressing said gene, under stringent conditions,
detecting the hybridization product of said probe and said cell DNA.

91. A method for in vitro detection of a deficient transcriptional activity of genes involved in the control of cell growth, cell differentiation, cell proliferation or cell apoptosis, comprising the step of detecting a deficient production of the transcript of said gene which would normally encode a polypeptide capable of binding an IPCS sequence and as a result would positively regulate the transcription of a nucleotide sequence placed under the control of said IPCS sequence.

92. A method for in vitro prognosis of transformation of cells toward a malignant state, which comprises the step of detecting a mutation in the PRDII-BFI gene normally expressed in said cells, or detecting a mutation in the transcript obtained by splicing of said gene, which mutation would result in lack of expression or in an abnormal expression of polypeptide expression product of said PRDII-BF1 gene capable of binding to an IPCS sequence.

93. A method for screening compounds for activity of regulating the transcriptional activity of genes containing an IPCS sequence in their promoter region, said genes being active in the control of cell growth, cell differentiation, cell proliferation, or cell apoptosis, comprising the steps of:
contacting said compound with DNA of a cell expressing genes containing an IPCS sequence in their promoter region,
detecting formation of a DNA-compound complex and assaying its transcriptional activity on said gene containing the IPCS sequence.

94. A method of screening compounds for activity as an agonist of GAAP-1 or of GAAP-2 comprising contacting said compound with a polypeptide of claim 65 and detecting the formation of a complex of said compound and said polypeptide.

95. A method of screening compounds for activity as an antagonist of GAAP-1 or of GAAP-2 comprising contacting said compound with a polypeptide of claim 65 and detecting the formation of a complex of said compound and said polypeptide.


SEQUENCE LISTING (48 sequences)

SEQ ID NO: 1 - DNA, 13 bp, Homo sapiens
aaatgrykkcmms

SEQ ID NO: 2 - DNA, 10 bp, Homo sapiens
  misc_feature (6)..(9): n is a, c, g, or t(u)
aaatgnnnnc

SEQ ID NO: 3 - DNA, 14 bp, Homo sapiens
aaaatgatttccac

SEQ ID NO: 4 - DNA, 14 bp, Homo sapiens
gaaatgacggcacg

SEQ ID NO: 5 - DNA, 10 bp, Homo sapiens
aaatgtattc

SEQ ID NO: 6 - DNA, 10 bp, Homo sapiens
aaatgaaaac

SEQ ID NO: 7 - DNA, 10 bp, Homo sapiens
aaatggtgac

SEQ ID NO: 8 - DNA, 10 bp, Homo sapiens
aaatgtgtcc

SEQ ID NO: 9 - DNA, 10 bp, Homo sapiens
aaatggcaac

SEQ ID NO: 10 - DNA, 10 bp, Homo sapiens
aaatgggctc

SEQ ID NO: 11 - DNA, 10 bp, Homo sapiens
aaatggtgcc

SEQ ID NO: 12 - DNA, 10 bp, Homo sapiens
aaatgaaggc

SEQ ID NO: 13 - DNA, 10 bp, Homo sapiens
aaatgaacac

SEQ ID NO: 14 - DNA, 10 bp, Homo sapiens
aaatgtcaac

SEQ ID NO: 15 - DNA, 10 bp, Homo sapiens
aaatgrykkc

SEQ ID NO: 16 - DNA, 14 bp, Artificial Sequence
  Description of Artificial Sequence oligonucleotide
aaaatgatttccac

SEQ ID NO: 17 - DNA, 14 bp, Artificial Sequence
  Description of Artificial Sequence oligonucleotide
aaaacgatttccac

SEQ ID NO: 18 - DNA, 14 bp, Artificial Sequence
  Description of Artificial Sequence oligonucleotide
gaaatgacggcacg

SEQ ID NO: 19 - DNA, 31 bp, Artificial Sequence
  Description of Artificial Sequence oligonucleotide
agcatggcattaggtaatcaaaagtccacag

SEQ ID NO: 20 - DNA, 22 bp, Artificial Sequence
  Description of Artificial Sequence oligonucleotide
ccatcaggttgctatcacaagc

SEQ ID NO: 21 - DNA, 20 bp, Artificial Sequence
  Description of Artificial Sequence oligonucleotide
accatggtgagcaagggcga

SEQ ID NO: 22 - DNA, 29 bp, Artificial Sequence
  Description of Artificial Sequence oligonucleotide
gattacctaatgctctcttgtacagctcg

SEQ ID NO: 23 - DNA, 36 bp, Artificial Sequence
  Description of Artificial Sequence oligonucleotide
ggacgagctgtacaagatggggcagaagtttcaaaa

SEQ ID NO: 24 - DNA, 22 bp, Artificial Sequence
  Description of Artificial Sequence oligonucleotide
ccatcaggttgctatcacaagc

SEQ ID NO: 25 - DNA, 35 bp, Artificial Sequence
  Description of Artificial Sequence oligonucleotide
ccttaatcaaaagtgaagatggaggatataagtca

SEQ ID NO: 26 - DNA, 31 bp, Artificial Sequence
  Description of Artificial Sequence oligonucleotide
atcctccatcttcacttttgattaaggaatt

SEQ ID NO: 27 - DNA, 31 bp, Artificial Sequence
  Description of Artificial Sequence oligonucleotide
gcagaagaagctggaatacgttgtaagaaac

SEQ ID NO: 28 - DNA, 30 bp, Artificial Sequence
  Description of Artificial Sequence oligonucleotide
agcttcttctgcaatgtattttcctcttcc

SEQ ID NO: 29 - DNA, 38 bp, Artificial Sequence
  Description of Artificial Sequence oligonucleotide
gcaatgaagtccaaggcaggaagcaagaaatgtgtgga

SEQ ID NO: 30 - DNA, 36 bp, Artificial Sequence
  Description of Artificial Sequence oligonucleotide
tcctgccttggacttcattgcttttgtcagatttcc

SEQ ID NO: 31 - DNA, 33 bp, Artificial Sequence
  Description of Artificial Sequence oligonucleotide
agcctgatttccccgaaatgacggcacgcagcc

SEQ ID NO: 32 - DNA, 30 bp, Artificial Sequence
  Description of Artificial Sequence oligonucleotide
aatgcaggattcctccaaaatgatttccac

SEQ ID NO: 33 - DNA, 24 bp, Artificial Sequence
  Description of Artificial Sequence oligonucleotide
gatcctctggggattccccatgga

SEQ ID NO: 34 - protein, 693 aa, Homo sapiens
MALGNQKSTVVEFSNKDASEINSEQDKENSLIKSEPRRIKIFDGGYKSNEEYVYIRGRGRGKYICEECGIRCKKPSMLKKHIRTHTDVRPYHCTYCNFSFKTKGNLTKHMKSKAHSKKCVDLGISVGLIDEQDTEESDEKQRFSYERSGYDLEESDGPDEDDNENEDDDEDSQAESVLSATPSVTASPQHLPSRSSLQDPVSTDEDVRITDCFSGVHTDPMDVLPRALLTRMTVLSTAQSDYNRKTLSPGKARQRAARDENDTIPSVDTSRSPCHQMSVDYPESEEILRSSMAGKAVAITQSPSSVRLPPAAAEHSPQTAAGMPSVASPHPDPQEQKQQITLQPTPGLPSPHTHLFSHLPLHSQQQSRTPYNMVPVGGIHVVPAGLTYSTFVPLQAGPVQLTIPAVSVVHRTLGTHRNTVTEVSGTTNPAGVAELSSVVPCIPIGQIRVPGLQNLSTPGLQSLPSLSMETVNIVGLANTNMAPQVHPPGLALNAVGLQVLTANPSSQSSPAPQAHIPGLQILNIALPTLIPSVSQVAVDAQGAPEMPASQSKACETQPKQTSVASANQVSRTESPQGLPTVQRENAKKVLNPPAPAGDHARLDGLSKMDTEKAASANHVKPKPELTSIQGQPASTSQPLLKAHSEVFTKPSGQQTLSPDRQVPRPTGLPRRQPTVHFSDVSSDDDEDRLVIAT

SEQ ID NO: 35 - protein, 51 aa, Homo sapiens
CEECGIRCKKPSMLKKHIRTHTDVRPYHCTYCNFSFKTKGNLTKHMKSKAH

SEQ ID NO: 36 - protein, 51 aa, Homo sapiens
AEEAGIRCKKPSMLKKHIRTHTDVRPYHCTYCNFSFKTKGNLTKHMKSKAH

SEQ ID NO: 37 - protein, 50 aa, Homo sapiens
CEECGIRCKKPSMLKKHIRTHTDVRPYHCTYCNFSFKTKGNLTKAMKSKA

SEQ ID NO: 38 - DNA, 2154 bp, Homo sapiens
  misc_feature (1)..(2154): GAAP-1 SEQUENCE
atggcattaggtaatcaaaagtccacagtagttgaattcagcaataaagatgcctctgaaattaacagtgagcaagataaagaaaattccttaatcaaaagtgaaccaagaagaattaaaatatttgatggaggatataagtcaaatgaagagtatgtatatgtccgaggcaggggaggaggaaaatacatttgtgaagaatgtggaatacgttgtaagaaacctagcatgttaaagaaacacatacgaacccatacagatgtccgcccctaccactgcacttactgtaacttctcctttaagactaaaggaaatctgacaaaacacatgaagtccaaggcacatagcaagaaatgtgtggatttaggcgtctcagtaggtttaatagatgaacaggatacagaagaatcagatgaaaaacagagattcagttatgagcgatctggatatgatcttgaagaatctgatggcccagatgaggatgacaatgcagagattcagttatgagcgatctggatatgatcttgaagaatctgatggcccagatgaggatgacaatgaaaatgaagacgatgatgaggacagccaggctgaatcagtcctgtcagccacaccctcagtcacagctagcccgcagcaccttccatctagaagtagccttcaggaccctgtgagtactgacgaggatgtcaggatcaccgattgcttttctggggtacacacggacccaatggacgttctgcccagggcgctgctcaccagaatgactgtcctgagcacagcacagtctgactacaataggaagacactctctccggggaaggccaggcagcgtgctgcgagagatgaaaacgacacaattccgtctgtagacacttccaggtccccgtgtcatcagatgtctgtggactaccctgagtcagaagaaattctgagaagttctatggcaggaaaagctgttgctataacacagagcccatcatctgtaagacttcctcctgctgcagctgagcacagcccccagacagcagcggggatgccttctgtggcctcaccacatcctgaccctcaagaacagaagcagcaaataactctacagccgactccaggcttgccttctccccacactcatttgtttagccaccttcctttgcattcccagcagcaatcgaggacaccttataatatggttccagttggggggatccatgtggtacctgctggcctcacatactccacgtttgtgccccttcaggctggaccagtgcagctcacgatccctgctgtcagtgtcgttcacagaactttgggtactcataggaatacggtcacagaagtgtctggcactacaaaccctgctggagtggctgaattaagcagtgttgtgccatgtattcctatcggccaaatccgcgtgccaggccttcagaacctaagtaccccaggcttgcagtcactcccctcgttaagcatggaaaccgtcaatattgtaggcctagccaatacaaatatggccccacaagtccatccaccaggactggctctgaatgctgtcggactgcaggttctgactgcaaacccttcatcacaaagcagccccgcccctcaggcacacattccaggtctccagatcttgaacatagcattgcccaccttaatcccctcagtcagtcaagtagccgttgatgcacagggagctccggaaatgccagcttcccaaagcaaagcatgcgagacacaacccaagcagacttctgtagccagcgcaaaccaggtcagcaggaccgagtctcctcaggggttacctacagtccagcgggaaaatgcaaaaaaagttctgaatccacctgcccctgcaggtgaccatgcaaggcttgatggcctgagtaaaatggacacagagaaggctgcctcggcaaatcacgtgaagcccaagcctgaactcacttccatacagggccaaccagcgtccacgtcacaacctctgctgaaggcacattctgaagtttttacaaagccctcaggccagcagactctctctccagacagacaggttcccaggcccacagcactaccgcggaggcagcccactgtgcacttcagcgacgtgagcagcgatgatgacgaggacaggcttgtgatagcaacctgatg

SEQ ID NO: 39 - DNA, 2014 bp, Homo sapiens
  misc_feature (1)..(2014): PRDII SEQUENCE
atggcattaggtaatcaaaagtccacagtagttgaattcagcaataaagatgcctctgaaattaacagtgagcaagataaagaaaattccttaatcaaaagtgaaccaagaagaattaaaatatttgatggaggatataagtcaaatgaagagtatgtatatatccgaggcaggggaagaggaaaatacatttgtgaagaatgtggaatacgttgtaagaaacctagcatgttaaagaaacacatacgaacccatacagatgtccgcccctaccactgcacttactgtaacttctcctttaagactaaaggaaatctgacaaaacacatgaagtccaaggcacatagcaagaaatgtgtggatttaggcatctcagtaggtttaatagatgaacaggatacagaagaatcagatgaaaaaaaaatgaagacgatgatgaggacagccaggctgaatcagtcctgtcagccacaccctcagtcacagctagcccgcagcaccttccatctagaagtagccttcaggaccctgtgagtactgacgaggatgtcaggatcaccgattgcttttctggggtacacacggacccaatggacgttctgcccagggcgctgctcaccagaatgactgtcctgagcacagcacagtctgactacaataggaagacactctctccggggaaggccaggcagcgtgctgcgagagatgaaaacgacacaattccgtctgtagacacttccaggtccccgtgtcatcagatgtctgtggactaccctgagtcagaagaaattctgagaagttctatggcaggaaaagctgttgctataacacagagcccatcatctgtaagacttcctcctgctgcagctgagcacagcccccagacagcagcggggatgccttctgtggcctcaccacatcctgaccctcaagaacagaagcagcaaataactctacagccgactccaggcttgccttctccccacactcatttgtttagccaccttcctttgcattcccagcagcaatcgaggacaccttataatatggttccagttggggggatccatgtggtacctgctggcctcacatactccacgtttgtgccccttcaggctggaccagtgcagctcacgatccctgctgtcagtgtcgttcacagaactttgggtactcataggaatacggtcacagaagtgtctggcactacaaaccctgctggagtggctgaattaagcagtgttgtgccatgtattcctatcggccaaatccgcgtgccaggccttcagaacctaagtaccccaggcttgcagtcactcccctcgttaagcatggaaaccgtcaatattgtaggcctagccaatacaaatatggccccacaagtccatccaccaggactggctctgaatgctgtcggactgcaggttctgactgcaaacccttcatcacaaagcagccccgcccctcaggcacacattccaggtctccagatcttgaacatagcattgcccaccttaatcccctcagtcagtcaagtagccgttgatgcacagggagctccagaaatgccagcttcccaaagcaaagcatgcgagacacaacccaagcagacttctgtagccagcgcaaaccaggtcagcaggaccgagtctcctcaggggttacctacagtccagcgggaaaatgcaaaaaaagttctgaatccacctgcccctgcaggtgaccatgcaaggcttgatggcctgagtaaaatggacacagagaaggctgcctcggcaaatcacgtgaagcccaagcctgaactcacttccatacagggccaaccagcgtccacgtcacaacctctgctgaaggcacattctgaagtttttacaaagccctcaggccagcagactctctctccagacagacaggttcccaggcccacaggactaccgcggaggcagcccactgtgcacttcagcgacgtgagcagcgatgatgacgaggacaggcttgtgatagcaacctgatg

SEQ ID NO: 40 - protein, 693 aa, Homo sapiens
  PEPTIDE (1)..(693): GAAP1 POLYPEPTIDE
MALGNQKSTVVEFSNKDASEINSEQDKENSLIKSEPRRIKIFDGGYKSNEEYVYVRGRGGGKYICEECGIRCKKPSMLKKHIRTHTDVRPYHCTYCNFSFKTKGNLTKHMKSKAHSKKCVDLGVSVGLIDEQDTEESDEKQRFSYERSGYDLEESDGPDEDDNENEDDDEDSQAESVLSATPSVTASPQHLPSRSSLQDPVSTDEDVRITDCFSGVHTDPMDVLPRALLTRMTVLSTAQSDYNRKTLSPGKARQRAARDENDTIPSVDTSRSPCHQMSVDYPESEEILRSSMAGKAVAITQSPSSVRLPPAAAEHSPQTAAGMPSVASPHPDPQEQKQQITLQPTPGLPSPHTHLFSHLPLHSQQQSRTPYNMVPVGGIHVVPAGLTYSTFVPLQAGPVQLTIPAVSVVHRTLGTHRNTVTEVSGTTNPAGVAELSSVVPCIPIGQIRVPGLQNLSTPGLQSLPSLSMETVNIVGLANTNMAPQVHPPGLALNAVGLQVLTANPSSQSSPAPQAHIPGLQILNIALPTLIPSVSQVAVDAQGAPEMPASQSKACETQPKQTSVASANQVSRTESPQGLPTVQRENAKKVLNPPAPAGDHARLDGLSKMDTEKAASANHVKPKPELTSIQGQPASTSQPLLKAHSEVFTKPSGQQTLSPDRQVPRPTALPRRQPTVHFSDVSSDDDEDRLVIAT

SEQ ID NO: 41 - protein, 693 aa, Homo sapiens; identical to SEQ ID NO: 34
  PEPTIDE (1)..(693): PRDII polypeptide

SEQ ID NO: 42 - protein, 23 aa, Homo sapiens
MGQKFQKKKSYRLVLKELRNPLK

SEQ ID NO: 43 - protein, 16 aa, Homo sapiens
KSYRLVLKELRNPLKR

SEQ ID NO: 44 - DNA, 2648 bp, Homo sapiens
atggggcagaagtttcaaaaaaagagagcattaggtaatcaaaagtccacagtagttgaattcagcaataaagatgcctctgaaattaacagtgagcaagataaagaaaattccttaatcaaaagtgaaccaagaagaattaaaatatttgatggaggatataagtcaaatgaagagtatgtatatatccgaggcaggggaagaggaaaatacatttgtgaagaatgtggaatacgttgtaagaaacctagcatgttaaagaaacacatacgaacccatacagatgtccgcccctaccactgcacttactgtaacttctcctttaagactaaaggaaatctgacaaaacacatgaagtccaaggcacatagcaagaaatgtgtggatttaggcatctcagtaggtttaatagatgaacaggatacagaagaatcagatgaaaaacagagattcagttatgagcgatctggatatgatcttgaagaatctgatggcccagatgaggatgacaatgaaaatgaagacgatgatgaggacagccaggctgaatcagtcctgtcagccacaccctcagtcacagctagcccgcagcaccttccatctagaagtagccttcaggaccctgtgagtactgacgaggatgtcaggatcaccgattgcttttctggggtacacacggacccaatggacgttctgcccagggcgctgctcaccagaatgactgtcctgagcacagcacagtctgactacaataggaagacactctctccggggaaggccaggcagcgtgctgcgagagatgaaaacgacacaattccgtctgtagacacttccaggtccccgtgtcatcagatgtctgtggactaccctgagtcagaagaaattctgagaagttctatggcaggaaaagctgttgctataacacagagcccatcatctgtaagacttcctcctgctgcagctgagcacagcccccagacagcagcggggatgccttctgtggcctcaccacatcctgaccctcaagaacagaagcagcaaataactctacagccgactccaggcttgccttctccccacactcatttgtttagccaccttcctttgcattcccagcagcaatcgaggacaccttataatatggttccagttggggggatccatgtggtacctgctggcctcacatactccacgtttgtgccccttcaggctggaccagtgcagctcacgatccctgctgtcagtgtcgttcacagaactttgggtactcataggaatacggtcacagaagtgtctggcactacaaaccctgctggagtggctgaattaagcagtgttgtgccatgtattcctatcggccaaatccgcgtgccaggccttcagaacctaagtaccccaggcttgcagtcactcccctcgttaagcatggaaaccgtcaatattgtaggcctagccaatacaaatatggccccacaagtccatccaccaggactggctctgaatgctgtcggactgcaggttctgactgcaaacccttcatcacaaagcagccccgcccctcaggcacacattccaggtctccagatcttgaacatagcattgcccaccttaatcccctcagtcagtcaagtagccgttgatgcacagggagctccagaaatgccagcttcccaaagcaaagcatgcgagacacaacccaagcagacttctgtagccagcgcaaaccaggtcagcaggaccgagtctcctcaggggttacctacagtccagcgggaaaatgcaaaaaaagttctgaatccacctgcccctgcaggtgaccatgcaaggcttgatggcctgagtaaaatggacacagagaaggctgcctcggcaaatcacgtgaagcccaagcctgaactcacttccatacagggccaaccagcgtccacgtcacaacctctgctgaaggcacattctgaagtttttacaaagccctcaggccagcagactctctctccagacagacaggttcccaggcccacaggactaccgcggaggcagcccactgtgcacttcagcgacgtgagcagcgatgatgacgaggacaggcttgtgatagcaacctgatggattttattttttatttgctttttttttatataacacttaaaggtttctttgaaaaccctcctttccttaaagcacatttttctgacataaactcatgactaatctttgtgcaatcatgaacttttgaccaataattgttgttttgtgtcagctccagccatttttgtacatgttgtatagacaattgtgccttttaggagctttatgtttagaaactgtacagattgttgaatatctatatacataaaaatatattatatatgtatatgaaaaccaggtagttatttgtgtttagtaaggaaaacctgtcaaataaatcaaatgattaaattatatgttccactgttgaatataaattttatggctatggggcagagtttctgtgtataaattagtatgtaaactccatatttatgtattcatattagtctttgaaaatgggtctgtcctccttgtgtaagacagtaactttacacttcagacagattttctgtgttatgaaatgtttcagtaaaatattgtttactgacctttaaaaa

SEQ ID NO: 45 - DNA, 2151 bp, Homo sapiens
  misc_feature Complement((1)..(2151)): GAAP-2 coding sequence
atggggcagaagtttcaaaaaaagaaatcttacaggctggtgttaaaggaacttcggaatcccttaaagagagcattaggtaatcaaaagtccacagtagttgaattcagcaataaagatgcctctgaaattaacagtgagcaagataaagaaaattccttaatcaaaagtgaaccaagaagaattaaaatatttgatggaggatataagtcaaatgaagagtatgtatatatccgaggcaggggaagaggaaaatacatttgtgaagaatgtggaatacgttgtaagaaacctagcatgttaaagaaacacatacgaacccatacagatgtccgcccctaccactgcacttactgtaacttctcctttaagactaaaggaaatctgacaaaacacatgaagtccaaggcacatagcaagaaatgtgtggatttaggcatctcagtaggtttaatagatgaacaggatacagaagaatcagatgaaaaacagagattcagttatgagcgatctggatatgatcttgaagaatctgatggcccagatgaggatgacaatgaaaatgaagacgatgatgaggacagccaggctgaatcagtcctgtcagccacaccctcagtcacagctagcccgcagcaccttccatctagaagtagccttcaggaccctgtgagtactgacgaggatgtcaggatcaccgattgcttttctggggtacacacggacccaatggacgttctgcccagggcgctgctcaccagaatgactgtcctgagcacagcacagtctgactacaataggaagacactctctccggggaaggccaggcagcgtgctgcgagagatgaaaacgacacaattccgtctgtagacacttccaggtccccgtgtcatcagatgtctgtggactaccctgagtcagaagaaattctgagaagttctatggcaggaaaagctgttgctataacacagagcccatcatctgtaagacttcctcctgctgcagctgagcacagcccccagacagcagcggggatgccttctgtggcctcaccacatcctgaccctcaagaacagaagcagcaaataactctacagccgactccaggcttgccttctccccacactcatttgtttagccaccttcctttgcattcccagcagcaatcgaggacaccttataatatggttccagttggggggatccatgtggtacctgctggcctcacatactccacgtttgtgccccttcaggctggaccagtgcagctcacgatccctgctgtcagtgtcgttcacagaactttgggtactcataggaatacggtcacagaagtgtctggcactacaaaccctgctggagtggctgaattaagcagtgttgtgccatgtattcctatcggccaaatccgcgtgccaggccttcagaacctaagtaccccaggcttgcagtcactcccctcgttaagcatggaaaccgtcaatattgtaggcctagccaatacaaatatggccccacaagtccatccaccaggactggctctgaatgctgtcggactgcaggttctgactgcaaacccttcatcacaaagcagccccgcccctcaggcacacattccaggtctccagatcttgaacatagcattgcccaccttaatcccctcagtcagtcaagtagccgttgatgcacagggagctccagaaatgccagcttcccaaagcaaagcatgcgagacacaacccaagcagacttctgtagccagcgcaaaccaggtcagcaggaccgagtctcctcaggggttacctacagtccagcgggaaaatgcaaaaaaagttctgaatccacctgcccctgcaggtgaccatgcaaggcttgatggcctgagtaaaatggacacagagaaggctgcctcggcaaatcacgtgaagcccaagcctgaactcacttccatacagggccaaccagcgtccacgtcacaacctctgctgaaggcacattctgaagtttttacaaagccctcaggccagcagactctctctccagacagacaggttcccaggcccacaggactaccgcggaggcagcccactgtgcacttcagcgacgtgagcagcgatgatgacgaggacaggcttgtgatagcaacctga

SEQ ID NO: 46 - protein, 701 aa, Homo sapiens
MGQKFQKKRALGNQKSTVVEFSNKDASEINSEQDKENSLIKSEPRRIKIFDGGYKSNEEYVYIRGRGRGKYICEECGIRCKKPSMLKKHIRTHTDVRPYHCTYCNFSFKTKGNLTKHMKSKAHSKKCVDLGISVGLIDEQDTEESDEKQRFSYERSGYDLEESDGPDEDDNENEDDDEDSQAESVLSATPSVTASPQHLPSRSSLQDPVSTDEDVRITDCFSGVHTDPMDVLPRALLTRMTVLSTAQSDYNRKTLSPGKARQRAARDENDTIPSVDTSRSPCHQMSVDYPESEEILRSSMAGKAVAITQSPSSVRLPPAAAEHSPQTAAGMPSVASPHPDPQEQKQQITLQPTPGLPSPHTHLFSHLPLHSQQQSRTPYNMVPVGGIHVVPAGLTYSTFVPLQAGPVQLTIPAVSVVHRTLGTHRNTVTEVSGTTNPAGVAELSSVVPCIPIGQIRVPGLQNLSTPGLQSLPSLSMETVNIVGLANTNMAPQVHPPGLALNAVGLQVLTANPSSQSSPAPQAHIPGLQILNIALPTLIPSVSQVAVDAQGAPEMPASQSKACETQPKQTSVASANQVSRTESPQGLPTVQRENAKKVLNPPAPAGDHARLDGLSKMDTEKAASANHVKPKPELTSIQGQPASTSQPLLKAHSEVFTKPSGQQTLSPDRQVPRPTGLPRRQPTVHFSDVSSDDDEDRLVIAT

SEQ ID NO: 47 - protein, 716 aa, Homo sapiens
MGQKFQKKKSYRLVLKELRNPLKRALGNQKSTVVEFSNKDASEINSEQDKENSLIKSEPRRIKIFDGGYKSNEEYVYIRGRGRGKYICEECGIRCKKPSMLKKHIRTHTDVRPYHCTYCNFSFKTKGNLTKHMKSKAHSKKCVDLGISVGLIDEQDTEESDEKQRFSYERSGYDLEESDGPDEDDNENEDDDEDSQAESVLSATPSVTASPQHLPSRSSLQDPVSTDEDVRITDCFSGVHTDPMDVLPRALLTRMTVLSTAQSDYNRKTLSPGKARQRAARDENDTIPSVDTSRSPCHQMSVDYPESEEILRSSMAGKAVAITQSPSSVRLPPAAAEHSPQTAAGMPSVASPHPDPQEQKQQITLQPTPGLPSPHTHLFSHLPLHSQQQSRTPYNMVPVGGIHVVPAGLTYSTFVPLQAGPVQLTIPAVSVVHRTLGTHRNTVTEVSGTTNPAGVAELSSVVPCIPIGQIRVPGLQNLSTPGLQSLPSLSMETVNIVGLANTNMAPQVHPPGLALNAVGLQVLTANPSSQSSPAPQAHIPGLQILNIALPTLIPSVSQVAVDAQGAPEMPASQSKACETQPKQTSVASANQVSRTESPQGLPTVQRENAKKVLNPPAPAGDHARLDGLSKMDTEKAASANHVKPKPELTSIQGQPASTSQPLLKAHSEVFTKPSGQQTLSPDRQVPRPTGLPRRQPTVHFSDVSSDDDEDRLVIAT

SEQ ID NO: 48 - DNA, 14 bp, Homo sapiens
aaaacgatttccac